(19) United States
(12) Patent Application Publication
(10) Pub. No.: US 2026/0325081 A1
(43) Pub. Date: Oct. 22, 2026

(54) HEALTH EVENT PREDICTION USING HEARTBEAT INTERVALS FROM ELECTROCARDIOGRAM DATA

(71) Applicant: Medtronic, Inc., Minneapolis, MN (US)

(72) Inventors: Evan J. Stanelle, Shoreview, MN (US); Dana M. Soderlund, Plymouth, MN (US)

(21) Appl. No.: 19/101,446

(22) PCT Filed: Jul. 19, 2023
(86) PCT No.: PCT/US2023/028145

§ 371 Date: Feb. 5, 2025

Related U.S. Application Data
(60) Provisional application No. 63/370,761, filed on Aug. 8, 2022.

(51) Int. Cl. A61B 5/00; A61B 5/318; G16H 40/67; G16H 50/30

(52) U.S. Cl. CPC A61B 5/7275; A61B 5/318; G16H 40/67; G16H 50/30

(57) ABSTRACT

An example system includes a memory; and processing circuitry coupled to the memory and configured to: receive electrocardiogram (ECG) data of a patient, wherein the ECG data is generated by one or more sensing devices of the patient based on physiological signals of the patient sensed by the one or more sensing devices; obtain a plurality of heartbeat intervals from the ECG data; sample a plurality of points for each respective heartbeat interval of the plurality of intervals; determine a slow-moving average for each sample point parameter of respective heartbeat intervals during a. first period of time; determine a fast-moving average for each sample point parameter of respective heartbeat intervals during a second period of time, determine a difference between the slow-moving average and the fast-moving average for each sample point parameter; and determine a risk level of a health event for the patient based on the determined differences.

This application is an international application with provisional priority of U.S. Provisional Patent Application No. 63/370,761, filed 8 Aug. 2022, the entire contents of which is incorporated herein by reference.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect health events, such as episodes of cardiac arrhythmia or worsening of heart failure, based on the physiological signals. Example arrhythmia types include asystole, bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. The devices may store ECG and other physiological signal data collected during a time period including an episode as episode data. The devices may also store episode data quantifying the episodes, e.g., number and/or duration of episodes. The medical device may also store ECG and other physiological data for a time period as episode data in response to user input, e.g., from the patient or a caregiver.

### SUMMARY

In general, the disclosure describes techniques for determining a risk level of a health event based on changes, including sometimes subtle changes, in sinus rhythm over a period of time. In some examples, the techniques include a computing system receiving ECG data, obtaining a plurality of heartbeat intervals from the ECG data, sampling a plurality of points for each respective heartbeat interval of the plurality of intervals, determining a difference between the slow-moving average and the fast-moving average for each sample point parameter, and determining a risk level of a health event for the patient based on the determined differences.

Determining a risk level of a health event for the patient based at least in part on determining a difference between a slow-moving average and a fast-moving average for each sample point parameter for each respective heartbeat interval of the plurality of obtained heartbeat intervals from ECG data may provide for easier implementation on a device or server that may more efficiently determine a risk level of a health event by reducing parameters used to determine a risk level of a health event.

In addition, determining a risk level of a health event for the patient based at least in part on determining a difference between a slow-moving average and a fast-moving average for each sample point parameter for each respective heartbeat interval of the plurality of obtained heartbeat intervals from ECG data may improve patient outcomes through more accurate and accessible predictive monitoring. In addition, providing insight or suggestions for intervention based on the determined risk level of a health event determined using techniques of this disclosure, may provide for intervention and/or treatment of a patient before a serious health event occurs, which may reduce morbidity and improve patient care.

The system described herein, may remotely monitor ECG signals, such as with an ICM, identify changes in the ECG signal that indicate an acute health event is likely to occur in the near future, and generate an output so intervention may occur before the acute health event occurs, which would improve patient outcomes and reduce morbidity of upcoming acute health events.

In one example, a system comprises a memory; and processing circuitry coupled to the memory and configured to: receive electrocardiogram (ECG) data of a patient, wherein the ECG data is generated by one or more sensing devices of the patient based on physiological signals of the patient sensed by the one or more sensing devices; obtain a plurality of heartbeat intervals from the ECG data; sample a plurality of points for each respective heartbeat interval of the plurality of intervals; determine a slow-moving average for each sample point parameter of respective heartbeat intervals during a first period of time; determine a fast-moving average for each sample point parameter of respective heartbeat intervals during a second period of time, the second period of time being less than the first period of time; determine a difference between the slow-moving average and the fast-moving average for each sample point parameter; and determine a risk level of a health event for the patient based on the determined differences.

In another example, a method comprises receiving electrocardiogram (ECG) data of a patient, wherein the ECG data is generated by one or more sensing devices of the patient based on physiological signals of the patient sensed by the one or more sensing devices; obtaining a plurality of heartbeat intervals from the ECG data; sampling a plurality of points for each respective heartbeat interval of the plurality of intervals; determining a slow-moving average for each sample point parameter of respective heartbeat intervals during a first period of time; determining a fast-moving average for each sample point parameter of respective heartbeat intervals during a second period of time, the second period of time being less than the first period of time; determining a difference between the slow-moving average and the fast-moving average for each sample point parameter; and determining a risk level of a health event for the patient based on the determined differences.

In another example, a system comprises a memory, and processing circuitry coupled to the memory and configured to: receive electrocardiogram (ECG) data of a patient, wherein the ECG data is generated by one or more sensing devices of the patient based on physiological signals of the patient sensed by the one or more sensing devices; obtain a plurality of heartbeat data from the ECG data; determine a slow-moving metric based on the heartbeat data during a first period of time; determine a fast-moving metric based on the heartbeat data during a second period of time, the second period of time being less than the first period of time; and determine a risk level of a health event for the patient based on the slow-moving metric and the fast-moving metric.

In another example, a system comprises processing circuitry configured to receive parametric data for a plurality of parameters of a patient. The parametric data is generated by one or more sensing devices of the patient based on physiological signals of the patient sensed by the one or more devices. The processing circuitry is further configured to determine a training set of parametric data for training a model based on the parametric data of the patient, classify the training set of parametric data based on classification data collected automatically in response to detection of a trigger, and train the model with the classified training set of parametric data.

In another example, a method comprises receiving parametric data for a plurality of parameters of a patient. The parametric data is generated by one or more sensing devices of the patient based on physiological signals of the patient sensed by the one or more devices. The method further comprises determining a training set of parametric data for training a model based on the parametric data of the patient, classifying the training set of parametric data based on classification data collected automatically in response to detection of a trigger, and training the model with the classified training set of parametric data.

In another example, a system comprises processing circuitry configured to perform any of the methods described herein.

In another example, a non-transitory computer readable storage medium comprises program instructions configured to cause processing circuitry to perform any of the methods described herein.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example medical device system configured to predict health events, and to respond to such predictions, in accordance with one or more techniques of this disclosure.

FIG. 2 is a block diagram illustrating an example configuration of the IMD of FIG. 1.

FIG. 3 is a conceptual side-view diagram illustrating an example configuration of the IMD of FIGS. 1 and 2.

FIG. 4 is a block diagram illustrating an example configuration of an external device that operates in accordance with one or more techniques of the present disclosure.

FIG. 5 is a block diagram illustrating an example computing system that operates in accordance with one or more techniques of the present disclosure.

FIG. 6 is a flow diagram illustrating an example technique for training a machine learning model using training sets of parametric data classified based on automatically collected classification data.

FIG. 7 is a flow diagram illustrating an example technique for automatically collecting classification data.

FIG. 8 is a flow diagram illustrating an example technique for predicting a health event and responding to the prediction of the health event.

FIG. 9 is a graph illustrating parametric data of a plurality of patient parameters over a time period around a stroke event.

FIG. 10 is a graph illustrating timeseries values of moving averages of parametric data of a patient parameter.

FIG. 11 is a chart illustrating experimentally-determined statistical significances of a plurality of patient parameters in predicting stroke.

FIG. 12 is a chart illustrating experimentally-determined statistical significances of a plurality of patient parameters in predicting stroke.

FIGS. 13A-13D are charts illustrating experimentally-determined statistical significances of a plurality of patient parameters in predicting stroke for different patient populations.

FIGS. 14A-14D are charts illustrating experimentally-determined statistical significances of a plurality of patient parameters in predicting hospitalization for different patient populations.

FIG. 15 is a chart illustrating AF burden pattern features for predicting stroke and health care utilization.

FIGS. 16A and 16B are diagrams illustrating AF burden patterns in patients who experience stroke or a health care utilization event, respectively, for various patient populations.

FIG. 17 is a graph illustrating detected AT/AF time (burden) over the course of a monitoring period.

FIG. 18 presents a scatterplot of terminal nodes by labeled HCU rate and percent of patients for balanced training data.

FIG. 19 presents an example graphical illustration of patterns in AF burden data mapped for a single HCU patient.

FIG. 20 presents a scatterplot of scored terminal nodes by labelled HCU rate and patient percent for an unbalanced validation set.

FIG. 21 presents a Venn diagram of AF burden threshold counts for the validation set.

FIG. 22 is a graph showing ECG data strips sampled nightly over a period of time in accordance with one or more techniques of the present disclosure.

FIG. 23 is a graph illustrating four heartbeat intervals sampled from the ECG data strips shown in FIG. 22 in accordance with one or more techniques of the present disclosure.

FIG. 24 is a graph illustrating the respective sampled heartbeat intervals shown in FIG. 23 normalized in length in accordance with one or more techniques of the present disclosure.

FIG. 25 is a graph illustrating a value of fast-moving average (FMA), a slow-moving average (SLMA), and a value of a sample point, of the sampled intervals shown in FIG. 24, over a period of time, a difference between the FMA and the SLMA of the sample point, and a risk threshold over the period of time in accordance with one or more techniques of the present disclosure.

FIGS. 26-29 are graphs illustrating sample point averages over a period of time of flagged sample intervals in accordance with one or more techniques of the present disclosure.

FIG. 30 is a flow diagram illustrating an example technique for predicting a health event in accordance with one or more techniques of the present disclosure.

FIG. 31 is a chart illustrating methodology for rare event prediction using continuous monitoring data in accordance with one or more techniques of the present disclosure.

Like reference characters refer to like elements throughout the figures and description.

### DETAILED DESCRIPTION

A variety of types of implantable and medical devices detect arrhythmia episodes (e.g., atrial fibrillation) and other health events based on sensed ECGs and, in some cases, other physiological signals. External devices that may be used to non-invasively sense and monitor ECGs and other physiological signals include wearable devices with electrodes configured to contact the skin of the patient, such as patches, watches, or necklaces. Such external devices may facilitate relatively longer-term monitoring of patient health during normal daily activities.

Implantable medical devices (IMDs) also sense and monitor ECGs and other physiological signals, and detect health events such as arrhythmia episodes and worsening heart failure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ Insertable Cardiac Monitor (ICM), available from Medtronic ple, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

Acute heath events, such a stroke and sudden cardiac arrest (SCA), have significant morbidity once they occur. This disclosure describes techniques of a system remotely monitoring ECG signals, such as with an ICM, identifying changes in the ECG signal that indicate an acute health event is likely to occur in the near future, and generating an output so intervention may occur before the acute health event occurs, which would improve patient outcomes and reduce morbidity of upcoming acute health events. In some examples, the near future may be within 5 to 10 days. In some examples, the near future may be a variety of other time periods, either greater than or less than 5 to 10 days.

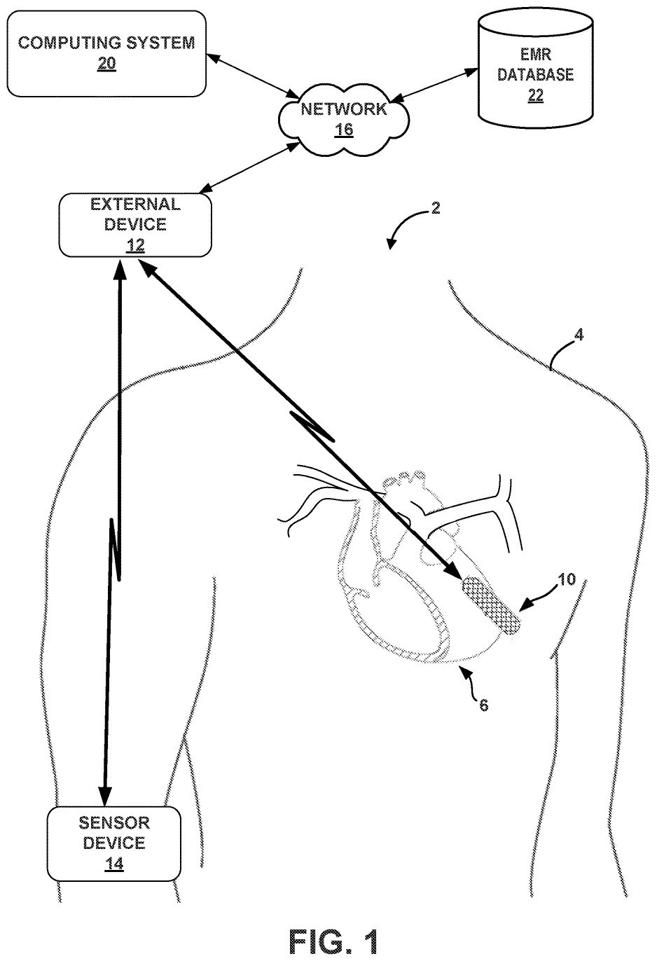

FIG. 1 is a block diagram illustrating an example medical device system 2 configured to predict health events of a patient 4, and to respond to such predictions, in accordance with the techniques of the disclosure. The example techniques may be used with an IMD 10, which may be in wireless communication with an external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense an ECG via the plurality of electrodes. In some examples, IMD 10 takes the form of the LINQ™ ICM. Although described primarily in the context of examples in which the IMD takes the form of an ICM, the techniques of this disclosure may be implemented in systems including any one or more implantable or external medical devices, including monitors, pacemakers, or defibrillators.

External device 12 is a computing device configured for wireless communication with IMD 10. External device 12 retrieves episode and other physiological data from IMD 10 that was collected and stored by IMD 10. In some examples, external device takes the form of a personal computing device of the patient or caregiver, such as a smartphone.

In the example illustrated by FIG. 1, system 2 also includes a sensor device 14 in wireless communication with external device 12. Sensor device 14 may include electrodes and other sensors to sense physiological signals of patient 4, and may collect and store physiological data and detect episodes based on such signals. In some examples, sensor device 14 is an external device wearable by patient 4. Sensor device 14 may be incorporated into the apparel of patient 14, such as within clothing, shoes, eyeglasses, a watch or wristband, a hat, etc. In some examples, sensor device 14 is a smartwatch or other accessory or peripheral for a smartphone external device 12.

External device 12 retrieves episode and other physiological data from sensor device 14 that was collected and stored by sensor device 14. External device 12 may include a display and other user interface elements. In some examples, external device 12 presents physiological data retrieved from IMD 10 and/or sensor device 14, and/or statistical representations thereof, to patient 4 or another user. External device 12 may communicate with IMD 10 and/or sensor device 14 according to the Bluetooth® or Bluetooth® Low Energy (BLE) protocols, as examples.

External device 12 may be configured to communicate with a computing system 20 via a network 16. External device 12 may be used to retrieve data from IMD 10 and sensor device 14, and may transmit the data to computing system 20 via network 16. The retrieved data may include values of physiological parameters measured by IMD 10 and sensor device 14, data regarding episodes of arrhythmia or other health events detected by IMD 10 and sensor device 14, and other physiological signals or data recorded by IMD 10 sensor device 14. The data retrieved from IMD 10 and sensor device 14 may include values of various patient parameters, and/or may be used by computing system 20 to determine values of patient parameters. The values of patient parameters may be referred to as patient parametric data. Patient parametric data may be retrieved and or determined on a periodic basis to produce periodic values, e.g., on a daily basis to produce daily values.

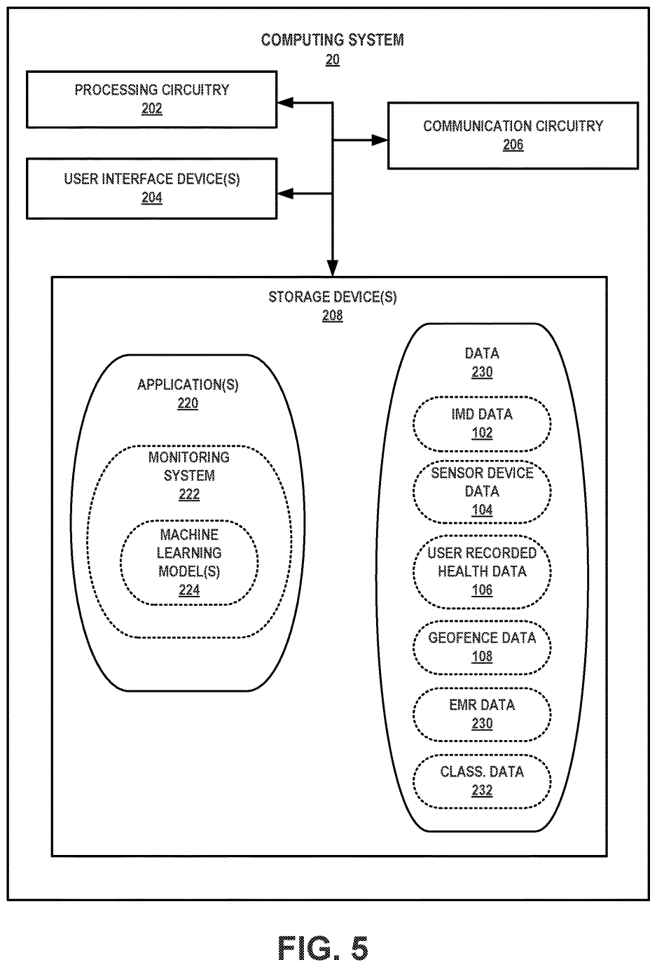

Computing system 20 may comprise computing devices configured to allow users, e.g., clinicians treating patient 4 and other patients, to interact with data collected from IMDs 10 and sensor devices 14 of their patients. In some examples, computing system 20 includes one or more handheld computing devices, computer workstations, servers or other networked computing devices. In some examples, computing system 20 may include one or more devices, including processing circuitry and storage devices, that implement a monitoring system 222 (FIG. 5).

In some examples, computing system 20 may receive ECG data, via IMD 10, obtain a plurality of heartbeat intervals from the ECG data over a period of time, sample or summarize a plurality of points for each respective heartbeat interval from the plurality of intervals, determine a difference between the slow-moving average and the fast-moving average for each sample point parameter, and determine a risk level of a health event for the patient based on the determined differences. In some examples, each obtained heartbeat interval from the ECG data may be a single heartbeat interval or an average of a plurality of adjacent and/or neighboring heartbeats from the ECG data, such as an average of 10 adjacent or neighboring heartbeats.

Monitoring system 222 may present parametric data of patients to clinicians to allow clinicians to remotely track and evaluate their patients. In some examples, monitoring system 222 may analyze the data and prioritize presentation of data or alerts for certain patients based on the analysis. Computing system 20, network 16, and monitoring system 222 may be implemented by the Medtronic Carelink™ Network, in some examples.

Network 16 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 16 may include one or more networks administered by service providers, and may thus form part of a large-scale public network infrastructure, e.g., the Internet. Network 16 may provide computing devices, such as computing system 20 and external device 12, access to the Internet, and may provide a communication framework that allows the computing devices to communicate with one another. In some examples, network 16 may be a private network that provides a communication framework that allows computing system 20 and external device 12 to communicate with one another but isolates one or more of these devices or data flows between these devices from devices external to network 16 for security purposes. In some examples, the communications between computing system 20 and external device 12 are encrypted.

Computing system 20 may also retrieve data for patient 4 from electronic medical records (EMR) database 22. EMR database 22 may store electronic medical records, also referred to as electronic health records, for patient 4, which may be generated by various health care providers, laboratories, clinicians, insurance companies, etc. Although illustrated as a single database in FIG. 1, EMR database 22 may include various databases managed by various entities.

As examples, EMR database 22 may store a medication history of the patient, a surgical procedure history of the patient, a hospitalization history of the patient, emergency or urgent care visit history of the patient, scheduled clinic visit history of the patient, one or more lab or other clinical test results for patient 14, a cardiovascular history of patient 14, or co-morbidities of patient 14 such as atrial fibrillation, heart failure, or diabetes, as examples. As further examples, EMR database 22 may store medical images for patient 4, such as x-ray images, ultrasound images, echocardiograms, anatomical imagery, medical photographs, radiographic images, etc. The data stored in EMR database 22 may include the patient specific records for patient 4 and numerous other patients. In some examples, the data stored by EMR database 22 may include broader demographic information or population-type information for a plurality of patients.

Monitoring system 222, e.g., implemented by processing circuitry of computing system 20, may implement the techniques of this disclosure including developing an algorithm based on training sets of parametric data of a population of patients or subjects retrieved from IMDs 10 and external devices 14 of the population, and applying the algorithm to parametric data of an individual patient 4 to predict the occurrence of a clinically significant health event. In some examples, monitoring system trains one or more machine learning (ML) models for prediction of the health event. The output of the ML models for a particular patient may be a level of risk of the health event, a probability of the health event occurring within a certain time, and/or whether the risk or probability satisfies a threshold.

Example health events that may be predicted using the techniques of this disclosure include stroke, clinically significant AF requiring hospitalization or urgent care, and clinically significant episodes of symptomatic events, such as syncope or dizziness. Parametric data that may be useful for predicting such health events may include cardiac rhythm data, such as heart rate data and data related to atrial fibrillation (AF) or other arrhythmia episodes. AF data may include quantifications of AF, referred to as AF burden, as well as patterns of AF burden over a plurality of periods of time. Parametric data that may be useful for predicting such clinically significant health events may additionally or alternatively include patient activity data or any other patient data or signals described herein.

Monitoring system 222 may also utilize data from EMR database 22 and/or data entered by the patient or a caregiver via external device 12 in conjunction with the parametric data from IMD 10 or sensor device 14. In some examples, data from EMR database 22 and/or data entered by the patient or caregiver may be used as inputs to the ML model(s) or other health event prediction algorithms implemented by monitoring system 222. In some examples, data from EMR database 22 and/or data entered by the patient or caregiver via external device 12 may provide classifications for training sets of parametric data from IMD 10 and sensor device 14 used to train one or more ML models to predict a health event. For example, data from EMR database 22 and/or data entered by the patient or caregiver via external device 12 may indicate whether, when, and to what degree of severity patient 4 experienced the clinically significant health event. Such data may be correlated with the parametric data to create a training set of parametric data. After an initial training phase, such training sets may be used for reinforcement learning and, in some cases, personalization of the one or more ML models.

Although the techniques are described herein as being performed by monitoring system 222, and thus by processing circuitry of computing system 20, the techniques may be performed by processing circuitry of any one or more devices or systems of a medical device system, such as computing system 20, external device 12, or IMD 10. The ML models may include, as examples, neural networks, deep learning models, convolutional neural networks, or other types of predictive analytics systems.

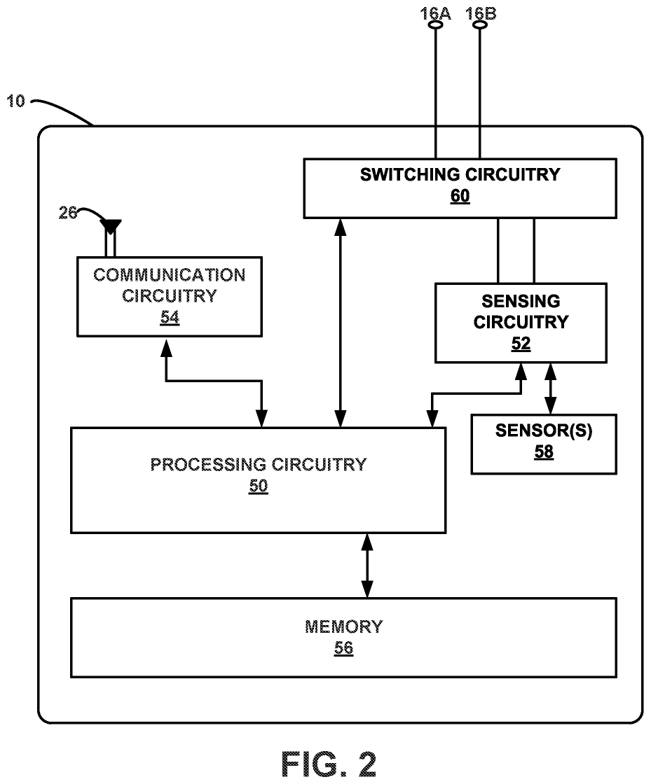

FIG. 2 is a block diagram illustrating an example configuration of IMD 10 of FIG. 1. As shown in FIG. 2, IMD 10 includes processing circuitry 50, sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor electrical activity of a heart of patient 4 of FIG. 1 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 56 as episode data for the detected arrhythmia episode. Processing circuitry 50 may also store parametric data in memory 56 including features of the ECG and data quantifying arrhythmia episodes, such as AF burden data.

Sensing circuitry 52 and/or processing circuitry 50 may be configured to detect cardiac depolarizations (e.g., P-waves of atrial depolarizations or R-waves of ventricular depolarizations) when the ECG amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and/or P-waves. Processing circuitry 50 may use the indications for determining features of the ECG including inter-depolarization intervals, heart rate, and heart rate variability. Sensing circuitry 52 may also provide one or more digitized ECG signals to processing circuitry 50 for analysis, e.g., for use in cardiac rhythm discrimination and/or to identify and delineate features of the ECG, such as QRS amplitudes and/or width, or other morphological features.

In some examples, sensing circuitry 52 measures impedance, e.g., of tissue proximate to IMD 10, via electrodes 16. The measured impedance may vary based on respiration and a degree of perfusion or edema. Processing circuitry 50 may determine parametric data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine parametric data, e.g., values of physiological parameters of patient 4, based on signals from sensors 58, which may be stored in memory 56.

In some examples, processing circuitry 50 transmits, via communication circuitry 54, the parametric and episode data for patient 4 to external device 12 of FIG. 1, which may transmit the data to network 16 for processing by monitoring system 222 of computing system 20. Communication circuitry 54 may include any suitable hardware, firmware, software or any combination thereof for communicating with another device, such as external device 12. Under the control of processing circuitry 50, communication circuitry 54 may receive downlink Telemetry from, as well as send uplink telemetry to, external device 12 or another device with the aid of an internal or external antenna, e.g., antenna 26.

Although described herein in the context of example IMD 10, the techniques for cardiac arrhythmia detection disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ICM, also commercially available from Medtronic PLC, a neurostimulator, or a drug delivery device.

As discussed with respect to FIG. 1, sensor device 14 may be an external device such as a smartwatch, a fitness tracker, patch, or other wearable device. Sensor device 14 may be configured similarly to IMD 10 in the sense that it may include electrodes, sensors, sensing circuitry, processing circuitry, memory, and communication circuitry, and may function similarly to collect parametric data and communicate with external device 12. The sensors of and parametric data collected by IMD 10 and sensor device 14 may differ as described herein.

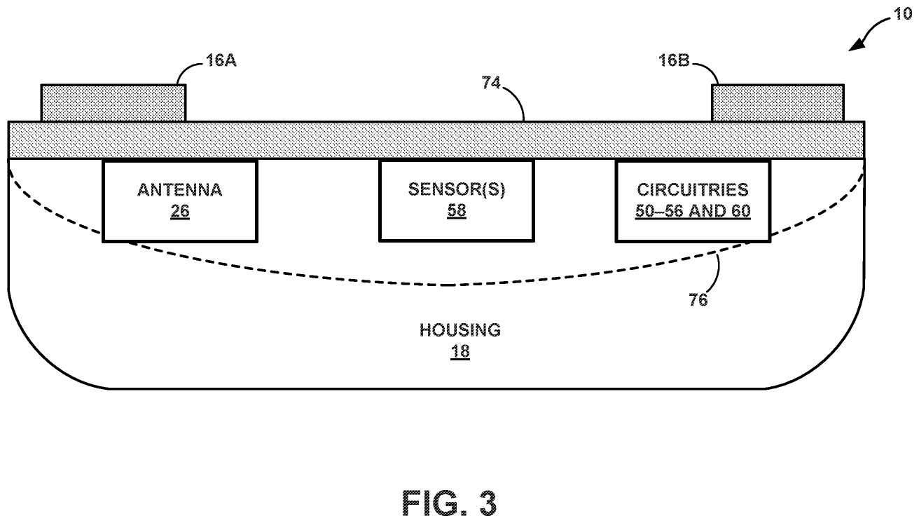

FIG. 3 is a conceptual side-view diagram illustrating an example configuration of IMD 10. In the example shown in FIG. 3, IMD 10 may include a leadless, subcutaneously-implantable monitoring device having a housing 18 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 18. In the illustrated example, antenna 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 18 such that housing 18 and cover 74 enclose antenna 26, sensors 58, and circuitries 50-56 and 60, and protect the antenna and circuitries from fluids such as body fluids.

One or more of antenna 26, sensors 58, or circuitries 50-56 may be formed on insulative cover 74, such as by using flip-chip technology. Insulative cover 74 may be flipped onto a housing 18. When flipped and placed onto housing 18, the components of IMD 10 formed on the inner side of insulative cover 74 may be positioned in a gap 76 defined by housing 18. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

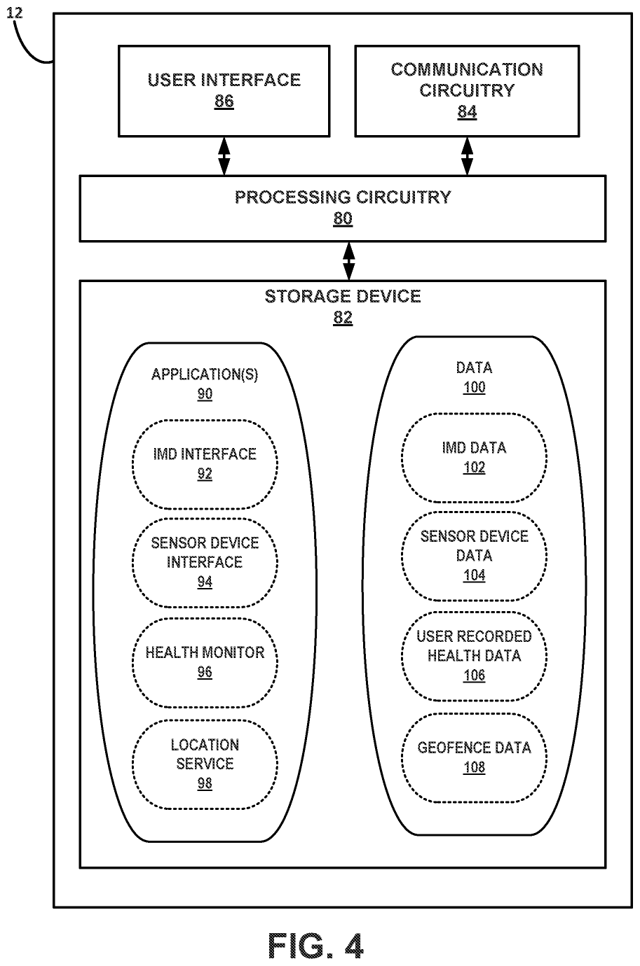

FIG. 4 is a block diagram illustrating an example configuration of external device 12. In some examples, external device 12 takes the form of a mobile device, such as a mobile phone, a “smart” phone, a laptop, a tablet computer, or a personal digital assistant (PDA). In some examples, external device 12 is a computing device of patient 4. As shown in the example of FIG. 4, external device 12 includes processing circuitry 80, storage device 82, communication circuitry 84, and a user interface 86. Although shown in FIG. 4 as a stand-alone device for purposes of example, external device 12 may be any component or system that includes processing circuitry or other suitable computing environment for executing software instructions and, for example, need not necessarily include one or more elements shown in FIG. 4 (e.g., in some examples components such as storage device 82 may not be co-located or in the same chassis as other components).

Processing circuitry 80, in one example, is configured to implement functionality and/or process instructions for execution within external device 12. For example, processing circuitry 80 may be capable of processing instructions, including applications 90, stored in storage device 82. Examples of processing circuitry 80 may include, any one or more of a microprocessor, a controller, a digital signal processor (DSP), an application specific integrated circuit (ASIC), a field-programmable gate array (FPGA), or equivalent discrete or integrated logic circuitry.

Storage device 82 may be configured to store information within external device 12, including applications 90 and data 100. Storage device 82, in some examples, is described as a computer-readable storage medium. In some examples, storage device 82 includes a temporary memory or a volatile memory. Examples of volatile memories include random access memories (RAM), dynamic random access memories (DRAM), static random access memories (SRAM), and other forms of volatile memories known in the art. Storage device 82, in one example, is used by applications 90 running on external device 12 to temporarily store information during program execution. Storage device 82, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

External device 12 utilizes communication circuitry 84 to communicate with other devices, such as IMD 10, sensor device 14, and computing system 20 of FIG. 1. Communication circuitry 84 may include a network interface card, such as an Ethernet card, an optical transceiver, a radio frequency transceiver, or any other type of device that can send and receive information. Other examples of such network interfaces may include 3G, 4G, 5G, and WiFi radios.

External device 12 also includes a user interface 86. User interface 86 may be configured to provide output to a user using tactile, audio, or video stimuli and receive input from a user through tactile, audio, or video feedback. User interface 86 may include, as examples, a presence-sensitive display, a mouse, a keyboard, a voice responsive system, video camera, microphone, or any other type of device for detecting a command from a user, a sound card, a video graphics adapter card, or any other type of device for converting a signal into an appropriate form understandable to humans or machines, a speaker, a cathode ray tube (CRT) monitor, a liquid crystal display (LCD), or any other type of device that can generate intelligible output to a user. In some examples, a presence-sensitive display includes a touch-sensitive screen.

Example applications 90 executable by processing circuitry 80 of external device 12 include an IMD interface application 92, a sensor device interface application 94, a health monitor application 96, and a location service 98. Execution of IMD interface 92 by processing circuitry 80 configures external device 12 to interface with IMD 10. For example, IMD interface 92 configures external device 12 to communicate with IMD 10 via communication circuitry 84. Processing circuitry 80 may retrieve IMD data 102 from IMD 10, and store IMD data 102 in memory 82. IMD interface 92 also configures user interface 86 for a user to interact with IMD 10 and/or IMD data 102. For example, IMD interface 92 configures external device 12 to communicate with IMD 10 via communication circuitry 84. Processing circuitry 80 may retrieve IMD data 102 from IMD 10, and store IMD data 102 in memory 82. IMD interface 92 also configures user interface 86 for a user to interact with IMD 10 and/or IMD data 102. Similarly, sensor device interface 94 configures external device 12 to communicate with sensor device 14 via communication circuitry 84, retrieve sensor device data 104 from sensor device 14, and store sensor device data 104 in memory 82. Sensor device interface 42 also configures user interface 86 for a user to interact with sensor device 14 and/or sensor device data 104.

Health monitor 96 may be configured facilitate monitoring the health of patient 4 by a user, such as the patient or a caregiver. Health monitor 96 may present health information, such as at least portions of IMD data 102 and/or sensor device data 104, via user interface 86. Health monitor 96 may also collect information regarding the patient's health from the user via user interface 86, and store the information as user recorded health data 106. In some examples, health monitor 96 present the user with a questionnaire or survey seeking health data 106 from the user. Health monitor 96 may present the surveys according to a schedule, in response to IMD data 102 and/or sensor device data 104 indicating that patient 4 experienced a health event, and/or based on a location of patient 4, e.g., in response to location service 98 indicating that patient 4 entered a geofence area defined by geofence data 108. Presenting surveys in response to health events may facilitate timely capture of user recorded health data 106 regarding the health event. In some examples, geofence areas are defined around clinics, hospitals, or the like, and entry into a such geofence area may similarly indicate that patient 4 experienced a health event meriting timely collection of user recorded health data 106. Processing circuitry 80 may also store the times and durations of patient entering a geofence area as geofence data 108.

IMD data 102 and sensor device data 104 may include patient parametric data derived from sensed physiological signals as described herein. As examples, IMD data 102 may include periodic (e.g., daily) values of one or more of: heart rate, heart rate variability, one or more ECG morphological features or intrabeat intervals, AF and/or other arrhythmia burden (e.g., number, time, or percent time per period), respiratory rate, perfusion, and activity levels.

As examples, sensor device data 104 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

As examples, user recorded health data 106 may include one or more of: exercise and activity data, sleep data, symptom data, medical history data, quality of life data, nutrition data, medication taking or compliance data, allergy data, demographic data, weight, and height. Symptom data may include times a patient experienced a symptom and their characterizations of the symptoms, such as palpitations, atrial flutter, AF, atrial tachycardia, syncope, or dizziness. Medical history data may relate to history of AF, stroke, chronic obstructive pulmonary disease (COPD), renal dysfunction, or hypertension, history of procedures, such as ablation or cardioversion, and healthcare utilization. Sensor device data 104 and/or user recorded health data 106 may include one or more of the types of data listed in Table 1 below.

TABLE 1

| | |
|---|---|
| Data Type | Relevance |
| Health Records (Allergies) | Could help explain why |
| | certain medications were |
| | not used |
| Health Records (Conditions) | Confirms patient medical conditions |
| Health Records (Lab results) | Confirm disease states |
| Health Records (Medications) | Confirm AF medication use |
| | (AAD, OAC, etc.) |
| Health Records (Procedures) | Tracks interventions that |
| | could impact AF |
| Health Records (Vital Signs) | Compare to IMD data |
| Activity | Compare to IMD data |
| Sex | |
| Date of Birth | |
| Walking + Running Distance | Compare to IMD data |
| Resting Energy | Compare to IMD data |
| Active Energy | Compare to IMD data |
| Exercise Minutes | Compare to IMD data |
| Stand Hour | Compare to IMD data |
| Stand Time | Compare to IMD data |
| Height | |
| Body Mass | |
| Body Mass Index | |
| Lean Body Mass | Greater understanding of |
| | body comp and fitness |
| | level compared to BMI |
| Body Fat Percentage | Greater understanding of |
| | body comp and fitness |
| | level compared to BMI |
| Waist Circumference | Greater understanding of |
| | body comp and fitness |
| | level compared to BMI |
| Heart Rate | Compare to IMD data |
| Low Heart Rate Notifications | Compare to IMD data |
| High Heart Rate Notifications | Compare to IMD data |
| Irregular Rhythm Notifications | Compare to IMD data |
| Resting Heart Rate | Compare to IMD data |
| Heart Rate Variability | Compare to IMD data |
| Walking Heart Rate Average | Compare to IMD data |
| Heart Beat Series | Compare to IMD data |
| ElectroCardiograms (ECG) | Compare to IMD data |
| Blood Oxygen | Can offer greater insight into |
| | COPD/HF/apnea |
| | influenced AF |
| Blood Pressure | Indicator of cardiovascular risk |
| Systolic Blood Pressure | Indicator of cardiovascular risk |
| Diastolic Blood Pressure | Indicator of cardiovascular risk |
| Respiratory Rate | Indicator of potential HF or |
| | COPD conditions that |
| | can impact AF |
| VO2 Max | Indicator of physical fitness, |
| | HIGHLY predictive of |
| | clinical outcomes |
| Blood Glucose | Indicates Diabetes management, |
| | driver of CHADS |
| | score and impacts clinical |
| | outcomes in AF patients |
| Insulin Delivery | Indicates Diabetes management, |
| | driver of CHADS |
| | score and impacts clinical outcomes |
| | in AF patients |
| Peripheral Perfusion Index | Indicator of HF or COPD that |
| | can influence AF |
| Sleep | Sleep patterns may influence AF |
| Dietary Energy | Can be associated with AF |
| Carbohydrates | Can be associated with AF |
| Fiber | Can be associated with AF |
| Dietary Sugar | Can be associated with AF |
| Total Fat | Can be associated with AF |
| Monounsaturation Fat | Can be associated with AF |
| Polyunsaturated Fat | Can be associated with AF |
| Saturated Fat | Can be associated with AF |
| Cholesterol | Can be associated with AF |
| Protein | Can be associated with AF |
| Calcium | Can be associated with AF |
| Potassium | Can be associated with AF |
| Sodium | Can be associated with AF |
| Caffeine | Can be associated with AF |
| Dizziness | Can be associated with AF |
| Fainting | Can be associated with AF |
| Fatigue | Can be associated with AF |
| Chest Tightness or Pain | Can be associated with AF |
| Raid, Pounding, or | Can be associated with AF |
| Fluttering Heartbeat | |
| Shortness of Breath | Can be associated with AF |
| Skipped Heartbeat | Can be associated with AF |
| Headache | Can be associated with AF |
| Night Sweats | Can be associated with AF |
| Sleep Changes | Can be associated with AF |
| Sleep Apnea | Can be associated with AF |
| Alcohol Use | Can be associated with AF |
| Drug Use | Can be associated with AF |
| Stress | Can be associated with AF |
| Anxiety | Can be associated with AF |
| Depression | Can be associated with AF |

FIG. 5 is a block diagram illustrating an example configuration of computing system 20. In the illustrated example, computing system 24 includes processing circuitry 202 for executing applications 220 that include monitoring system 222 or any other applications described herein. Computing system 20 may be any component or system that includes processing circuitry or other suitable computing environment for executing software instructions and, for example, need not necessarily include one or more elements shown in FIG. 5 (e.g., user interface devices 204, communication circuitry 206; and in some examples components such as storage device(s) 208 may not be co-located or in the same chassis as other components). In some examples, computing system 20 may be a cloud computing system distributed across a plurality of devices.

In the example of FIG. 5, computing system 24 includes processing circuitry 202, one or more user interface (UI) devices 204, communication circuitry 206, and one or more storage devices 208. Computing system 20, in some examples, further includes one or more application(s) 220 such as monitoring system 222, that are executable by computing system 20.

Processing circuitry 202, in one example, is configured to implement functionality and/or process instructions for execution within computing system 20. For example, processing circuitry 202 may be capable of processing instructions stored in storage device 208. Examples of processing circuitry 202 may include any one or more of a microprocessor, a controller, a digital signal processor (DSP), an application specific integrated circuit (ASIC), a field-programmable gate array (FPGA), or equivalent discrete or integrated logic circuitry.

One or more storage devices 208 may be configured to store information within computing device 20 during operation. Storage device 208, in some examples, is described as a computer-readable storage medium. In some examples, storage device 208 is a temporary memory, meaning that a primary purpose of storage device 208 is not long-term storage. Storage device 408, in some examples, is described as a volatile memory, meaning that storage device 408 does not maintain stored contents when the computer is turned off. Examples of volatile memories include random access memories (RAM), dynamic random access memories (DRAM), static random access memories (SRAM), and other forms of volatile memories known in the art. In some examples, storage device 208 is used by software or applications 220 running on computing system 20 to temporarily store information during program execution.

Storage devices 208 may further be configured for long-term storage of information, such as applications 220 and data 230. In some examples, storage devices 208 include non-volatile storage elements. Examples of such non-volatile storage elements include magnetic hard discs, optical discs, floppy discs, flash memories, or forms of electrically programmable memories (EPROM) or electrically erasable and programmable memories (EEPROM).

Computing system 20, in some examples, also includes communication circuitry 206 to communicate with other devices and systems, such as IMD 10 and external device 12 of FIG. 1. Communication circuitry 206 may include a network interface card, such as an Ethernet card, an optical transceiver, a radio frequency transceiver, or any other type of device that can send and receive information. Other examples of such network interfaces may include 3G, 4G, 5G, and WiFi radios.

Computing system 20, in one example, also includes one or more user interface devices 204. User interface devices 204, in some examples, may be configured to provide output to a user using tactile, audio, or video stimuli and receive input from a user through tactile, audio, or video feedback. User interface devices 204 may include, as examples, a presence-sensitive display, a mouse, a keyboard, a voice responsive system, video camera, microphone, or any other type of device for detecting a command from a user, a sound card, a video graphics adapter card, or any other type of device for converting a signal into an appropriate form understandable to humans or machines, a speaker, a cathode ray tube (CRT) monitor, a liquid crystal display (LCD), or any other type of device that can generate intelligible output to a user.

Applications 220 may also include program instructions and/or data that are executable by processing circuitry 202 of computing system 20 to cause computing system 20 to provide the functionality ascribed to it herein. Example application(s) 220 may include monitoring system 22. Other additional applications not shown may alternatively or additionally be included to provide other functionality described herein and are not depicted for the sake of simplicity.

In accordance with the techniques of the disclosure, computing system 20 receives IMD data 102, sensor device data 104, user recorded health data 106, and geofence data 108 from external device 12 via communication circuitry 206. Processing circuitry 202 stores these as data 230 in storage devices 208.

Computing system 20 may also receive EMR data 230 from EMR database 22 (FIG. 1) vis communication circuitry 206, and store EMR data 230 in storage device 208. EMR data 230 may include, for each of a plurality of patients or subjects a medication history, a surgical procedure history, a hospitalization history, emergency or urgent care visit history, scheduled clinic visit history, one or more lab or other clinical test results, a procedure history, a cardiovascular history, or co-morbidities such as atrial fibrillation, heart failure, syncope, or diabetes, as examples. As further examples, EMR data 230 may include medical images, such as x-ray images, ultrasound images, echocardiograms, anatomical imagery, medical photographs, radiographic images, etc.

Monitoring system 222, e.g., implemented by processing circuitry of computing system 20, may implement the techniques of this disclosure including developing an algorithm based on training sets of parametric data, e.g., from IMD data 102 and sensor device data 104, and in some cases user recorded health data 106 and EMR data 230, of a population of patients or subjects, and applying the algorithm to parametric data of an individual patient 4 to predict the occurrence of a clinically significant health event. In some examples, monitoring system 222 trains one or more machine learning (ML) models 224 for prediction of the health event. The output of the ML models for a particular patient may be a level of risk of the health event, e.g., a probability of the health event, a level of risk or probability of the health event occurring within a certain predetermined time period, and/or whether the risk or probability satisfies a threshold.

The plurality of patient parameters may include AF burden, one or more activity parameters, and/or any of the physiological parameters described herein. In some examples, monitoring system 222 may derive features from the parametric data, and apply the features as inputs to the algorithm, e.g., ML model 224, to determine the risk level. One or more of the features may be AF burden features.

One or more of the features may be AF burden pattern features. An AF burden pattern feature may quantify a pattern of AF burden over a plurality of periods including the current period for which monitoring system 222 is determining the risk level. AF burden patterns including a change, e.g., spike or increase, in AF relative to an overall AF burden trend may be associated with an increased risk of a health event, such as a stroke of other clinically significant episode related to cardiovascular health. In some examples, monitoring system 222 determines the AF burden pattern feature by comparing, e.g., determining a difference or ratio between, a current AF burden value and an average, e.g., mean or median, of previous AF burden values. The current value may be a single value for the current period of a shorter-term average of values including the current period and a number of preceding periods. The average value may be a longer-term average of previous values, e.g., including more values and/or values from further in the past, which may not include the current period value. In some examples, the features include a patient activity feature, such as a daily activity level, a daytime or nighttime activity level, or a change in such an activity level relative to a baseline or trend in activity levels.

In general, the health event may be any clinically significant health event. In some examples, the health event may be a cardiovascular event. The health event may be a stroke. In some examples, the health event is a health care utilization event, such as a hospitalization. In some examples, the health event comprises a symptomatic event, such as clinically significant syncope or dizziness.

Monitoring system 222 may receive ECG data of patient 4, which may be generated by one or more sensing devices, such as IMD 10, of the patient based on physiological signals of the patient sensed by the one or more sensing devices, such as IMD 10. Monitoring system 222 may obtain a plurality of heartbeat intervals from the ECG data over a period of time, sample or summarize a plurality of points for each respective heartbeat interval from the plurality of intervals, and normalize the plurality of sampled or summarized points to a common length for each respective heartbeat interval of the plurality of intervals. In some examples, each obtained heartbeat interval from the ECG data may be a single heartbeat interval or an average of a plurality of adjacent and/or neighboring heartbeats from the ECG data, such as, for example, an average of 10 adjacent or neighboring heartbeats.

Monitoring system 222 may determine a slow-moving average for each sample point parameter of respective heartbeat intervals using one period of time and determine a fast-moving average for each sample point parameter of respective heartbeat intervals using a second period of time. The second period of time of the fast-moving average is less than the first period of time of the slow-moving average. Monitoring system 222 may determine a difference between the slow-moving average and the fast-moving average for each sample point parameter and determine a risk level of a health event for the patient based on the determined differences.

In some examples, monitoring system 222 may determine a comparison value for each sample point parameter by comparing an absolute value of the difference between the slow-moving average and the fast-moving average for each sample point parameter to a difference threshold and determine the risk level of the health event for the patient based on the comparison values for each sample point parameter.

Monitoring system 222 may generate an output to indicate the determined risk level of the health event. In some examples, monitoring system 222 may generate suggested responses, such as treatment, changes in medication, suggestion to go to the hospital, to be output in response to determining the risk level of the health event. Accordingly, in some examples, due to monitoring system 222 determining the risk level of the health event for the patient, serious health events, such as stoke, MI, or SCA, may be treated before the health event occurs, which may provide greater care for a patient and/or reduce healthcare costs.

Monitoring system 222 may initially train ML model 224 with parametric data collected from one or more populations of patients, e.g., during a clinical study. In conventional clinical studies, one or more human experts review the parametric data and collect other information to classify each of the training sets by endpoint, e.g., as either including the health event or not. In contrast, monitoring system 222 may classify the training sets of parametric data based on classification data 232 collected automatically in response to detection of a trigger, which may reduce the cost or manpower overhead associated with the clinical study.

In some examples, processing circuitry 202 executing monitoring system 222 collects the classification data 232. In some examples, classification data is additionally or alternatively collected by other processing circuitry of system 2 (FIG. 1), such as processing circuitry 80 of external device 12 (FIG. 4), and received by computing system 20 from the other processing circuitry. Classification data 232 includes data indicative of an endpoint for the training set of parametric data, e.g., indicative of whether the patient experienced the health event or not. Classification data 232 may include data from user recorded health data 106, geofence data 108, and/or EMR data 230 indicative of an endpoint for a patient.

Any one or more of IMD 10, sensor device 14, external device 12, or computing system 20 may detect the trigger for collection of classification data 232. In some examples, the trigger is a geofence event, e.g., detected by external device 12 indicating that the patient went to a hospital or clinic for a threshold amount of time. In such examples, external device 12 or computing system 20 may present a survey to the patient to collect information regarding the visit, e.g., confirming the visit and regarding the health issue(s) addressed, as user recorded health data 106 and classification data 230.

In some examples, the trigger comprises a feature of the parametric data for the patient satisfying a criterion, e.g., indicating that the patient may have experienced the health event. For example, a trigger may be AF burden meeting or exceeding a threshold. Other example triggers may include a feature of any physiological parameter described herein meeting a threshold value. In some examples, the trigger feature may be included within a training set of features used to train ML model 224, e.g., a set of features from which monitoring system 222 may choose to be input features based on their predictive value for the health event. In response to the detection of the trigger feature, monitoring system 222 or other processing circuitry of system 2 may collect classification data 230. The collection of classification data 232 may be via a survey as discussed above, or by checking geofence data 108 and/or EMR data 230 to identify a time proximate hospital or clinic visit indicative of the occurrence of the health event.

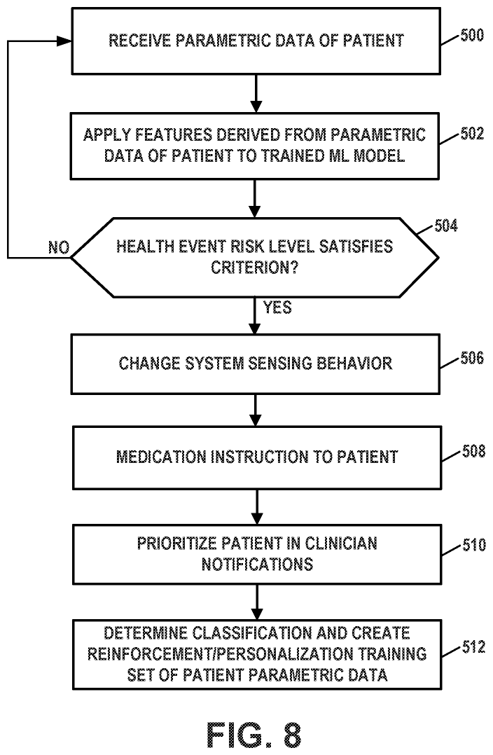

Subsequent to training ML model 224, monitoring system 222 may apply the ML model to parametric data, e.g., IMD data 102 and sensor device data 104, of a particular patient, such as patient 4, to determine a risk level that the patient with experience the health event. In some examples, monitoring system 222 may determine whether risk level of the health event satisfies a criterion, e.g., meets or exceeds a threshold risk level. Monitoring system 222 may take one or more actions based on determining that the risk level satisfied the criterion, e.g., as described with respect to FIG. 8.

Although the techniques are described herein as being performed by monitoring system 222, and thus by processing circuitry 202 of computing system 20, the techniques may be performed by processing circuitry of any one or more devices or systems of system 2. In some examples, external device 12 may additionally or alternatively implement monitoring system 222, e.g., using ML model 224 trained based on population parametric data and, in some examples, personalized based on parametric data of patient 4. ML model 224 may include, as examples, neural networks, deep learning models, convolutional neural networks, or other types of predictive analytics systems. Furthermore, although the techniques of this disclosure are described primarily with respect to examples including ML model 224, in some examples the techniques may be implemented with different models or algorithms that do not necessarily require machine learning, such as linear regression, trend analysis, decision trees, or thresholds, as examples.

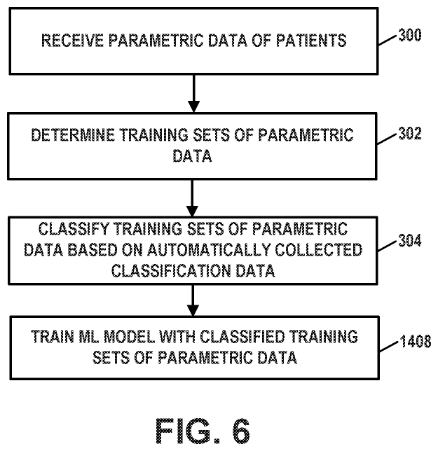

FIG. 6 is a flow diagram illustrating an example technique for training a machine learning model using training sets of parametric data classified based on automatically collected classification data. According to the example illustrated by FIG. 6, monitoring system 222 receives parametric data, such as IMD data 102 and sensor device data 104, of a plurality of patients (300). Monitoring system 222 determines training sets of the parametric data (302). Monitoring system 222 classifies the training sets of parametric data based on automatically collected classification data, as discussed above with reference to FIG. 5 (304). Monitoring system 222 trains ML model 224 with the classified training sets of parametric data (306)

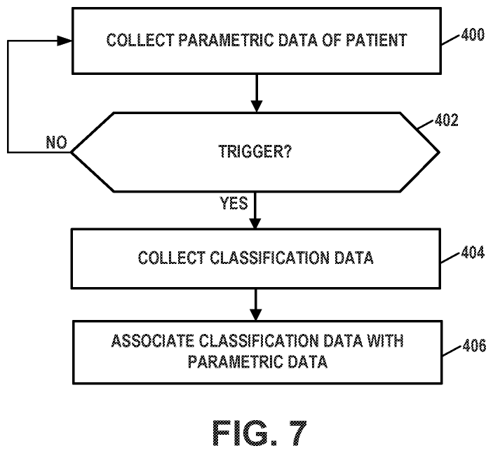

FIG. 7 is a flow diagram illustrating an example technique for automatically collecting classification data. According to the example of FIG. 7, monitoring system 222 collects parametric data of a patient, e.g., among a plurality of patient during a clinical study and ML model training phase (400). As discussed above with respect to FIG. 5, monitoring system determines whether trigger occurred (402). As discussed above with respect to FIG. 5, example triggers include a feature in the parametric data satisfying a criterion or a geofence event. A geofence event may be an event where a patient is within a geofenced area (e.g., an area near or around a hospital, urgent care clinic, and/or healthcare provider) for longer than a threshold time. The patient being with the geofenced area for longer than a threshold hold time may be evidence of unplanned or planned healthcare utilization. Examples of features in the parametric data satisfying a criterion include AF burden or other features derived from an ECG, e.g., heart rate or heart rate variability, exceeding a threshold, and/or a patient activity feature falling below a threshold. If the trigger did not occur (NO of 402), monitoring system 222 may continue to receive parametric data of the patient and monitor for the trigger (400, 402).

If the trigger occurs (YES of 402), monitoring system 222 collects classification data 232 (404). As discussed above with respect to FIG. 5, example classification data 232 may include user recorded health data 106 from a survey delivered to the patient in response to the trigger, or time proximate geofence data 108 (in the case of a parametric data feature trigger) or EMR data 230 indicating the patient visited a hospital or clinic and, in some cases, that the health event occurred. Monitoring system 222 associates the classification data 232 with the parametric data for eventual classification of a training set of parametric data (406).

FIG. 8 is a flow diagram illustrating an example technique for predicting a health event and responding to the prediction of the health event. As discussed above, example health events include stroke, hospitalization or other health care utilization, or symptomatic events, such as symptomatic AF or other cardiovascular events.

According to the example illustrated by FIG. 8, monitoring system 222 receives parametric data, e.g., IMD data 102 and sensor device data 104, for patient 4 (500). Monitoring system 222 applies features derived from the parametric data to ML model 224 (502). As discussed above, the features may include an AF feature, such as an AF burden pattern feature, and, in some cases, a patient activity feature or other feature derived from another physiological signal. Monitoring system 222 determines a risk level of the health event based on the application of the features to ML model 224, e.g., ML model 224 outputs a probability of the health event occurring with a predetermined period of time, such as a number of days. Monitoring system 222 determines whether the risk level of the health event satisfies a criterion, e.g., meets or exceeds a threshold (504). If the risk level does not satisfy the criterion (NO of 504), monitoring system 222 continues to receive parametric data and apply features to ML model 224, e.g., on a period by period basis (500, 502). Based on the risk level satisfying the criterion (YES of 504), monitoring system 222 may perform one or more of the optional actions illustrated by FIG. 8 (506-512).

Monitoring system 222 may change a sensing behavior of system 2 (506). For example, monitoring system 222 may direct IMD 10 and/or sensing device 104 to employ more sensitive setting for sensing circuitry 52 or sensors 58, sample physiological signals at a higher rate, and or make periodic measurements at a greater frequency.

As another example, monitoring system 222 may provide an instruction to patient 4 to take a medication or modify the taking of a medication (508). The medication may be an anticoagulant. The instruction may be to take a pro re nata dose of the medication or change a dosage of the medication.

As another example, monitoring system 222 may prioritize patient 4, or the portions of parametric data associated with the risk level of the health event, in a notification for a clinician treating patient 4 (810). Monitoring system 222 implementing the techniques of this disclosure may advantageously reduce the burden of treating patients by prioritizing patients and/or patient data in their notification from system 2 based on the risk level satisfying a criterion indicating a clinically significant risk of the health event. In some examples, monitoring system 222 reduces burden by determining which rhythms should be transmitted or alerted to the patient and/or clinician, e.g., presents a clinically relevant patient report that adjudicates symptoms.

As another example, monitoring system 222 may determine a classification for the parametric data associated with the risk level of the health event, and create a training set of parametric data for reinforcement training of ML model 224 and/or personalization of ML model 224 for patient 4, e.g., to create a patient-specific version of ML model 224 (512). Monitoring system 222 may utilize any of the techniques described herein, e.g., with respect to FIG. 7, to collect classification data 232 for classifying the training set of parametric data.

Health monitor 96 executed by processing circuitry 80 of external device may implement portions of the techniques described with respect to FIGS. 6-8. For example, health monitor 96 may present surveys and collect answers from patient 4, present instructions to take medication to patient 4, and provide enable messaging between patient 4 and a clinician.

In some examples, in order to enable real-time patient management, health monitor 96 can follow a pre-determined protocol to automatically push patient actions based on specific, detected patterns of parametric data. For example, health monitor 96 may see a predetermined clinically significant degree of AF burden and recommend modifications to a patient's anticoagulation medication. As discussed above, the actions may additionally or alternatively be pushed based on the risk level of the health event satisfying a criterion. In some examples, computing system 20 may provide an interface for a clinician via a web interface or user interface devices 204 to specify the parametric data features or risk level criterion that would trigger clinical action, e.g., AF duration lasting longer than 1 hour or probability of stroke exceeding a threshold probability, and the clinical action that the patient would need to take, such as an up titration of anticoagulation medications. In some examples, health monitor 96 may provide a pro re nata (PRN) medication request. In some examples, health monitor 96 may have a communication tab and also a priority status that would require the action to be acknowledged before allowing patient 4 to move on to other features of health monitor 96, such as viewing parametric data of patient 4.

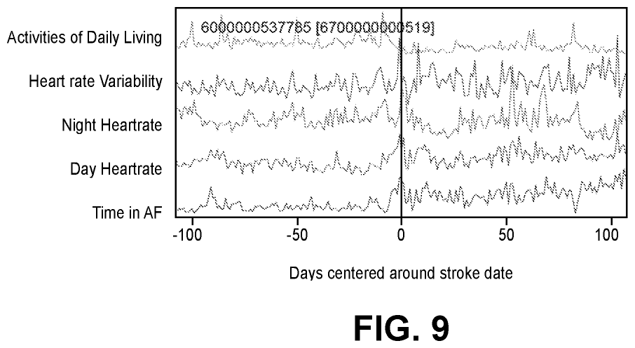

FIG. 9 is a graph illustrating parametric data of a plurality of patient parameters over a time period around a stroke event (time 0). In the example of FIG. 9, the patient parameters include a patient activity parameter (Activities of Daily Living, related to an amount of patient motion exceeding a threshold during daytime hours), heart rate variability (HRV), night heart rate, day heart rate, and time in AF (or AF burden). As can be seen in the illustrated example, time in AF and the heart rate related parameters all increase, and patient activity decreases, in the days leading up to the stroke.

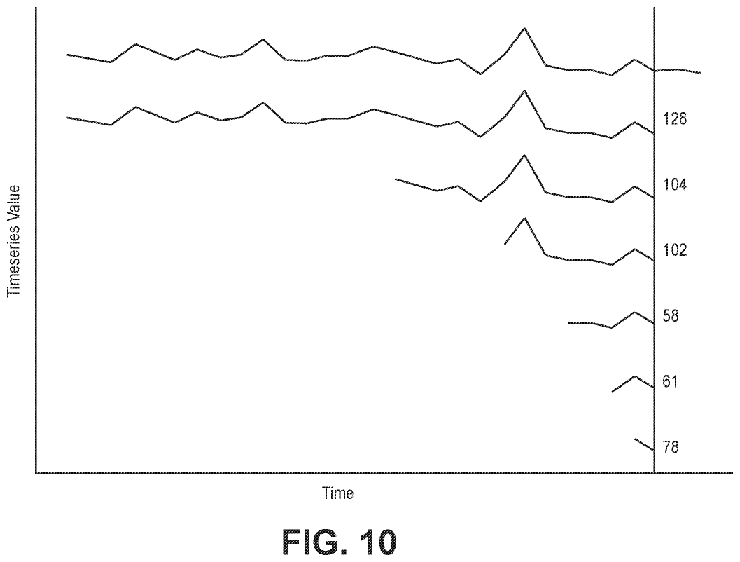

FIG. 10 is a graph illustrating timeseries values of moving averages of parametric data of a patient parameter. In the example illustrated by FIG. 10, the patient parameter is Activities of Daily Living, although similar techniques may be applied to any other patient parameter described herein. FIG. 10 illustrates a technique for quantifying a feature related to an excursion of a patient parameter from its baseline or trend, which may be indicative of an increased risk of the health event. In some examples, monitoring system 222 summarizes a trend with at least two simple moving averages (SMAs), and uses a comparison or offset of the two SMAs to capture a clinically significant change in the patient parameter. One SMA may be a shorter-term SMA and the other a longer-term SMA, e.g., that includes less recent values of the patient parameter than the shorter term SMA. Patient parameter values occurring within a predetermined number of days of the health event, e.g., stroke, may be identified. Under-sample controls may be 1:1 with cases, and all offsets and covariates may be evaluated in one model. Monitoring system 22 may compare goodness-of-fit for each variable (patient parameter) to determine relative importance of the variables.

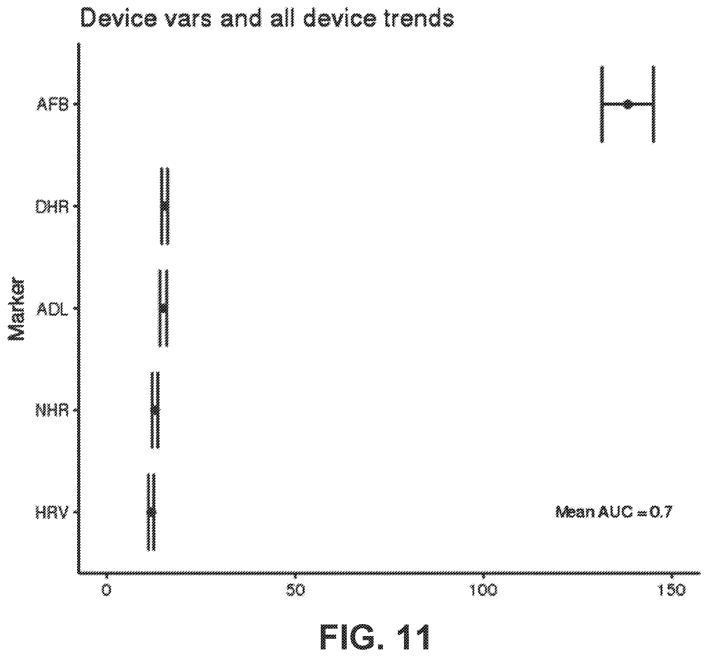

FIG. 11 is a chart illustrating experimentally-determined statistical significances of a plurality of patient parameters in predicting stroke. The patient parameters in the example of FIG. 11 are AF burden (AFB), day heart rate (DHR), activities of daily living (ADL), night heart rate (NHR), and heart rate variability (HRV). The statistical significances illustrated in FIG. 11 were determined based on parametric data collected from a plurality of patients including patients that suffered a stroke. As illustrated in FIG. 11, AF burden was found to be a significantly better predictor of stroke than the other patient parameters.

The experimental analysis suggested that a change in AF burden occurs within a long-term trend (21+days) prior to a stroke event. AF burden may be considered the leading predictor in the long-term. More particularly, a growing short-term trend in AF burden within a longer term trend may be predictive of stroke. The predictive ability of AF burden may be 4× greater when acute, shorter-term changes are compared to a longer-term trend.

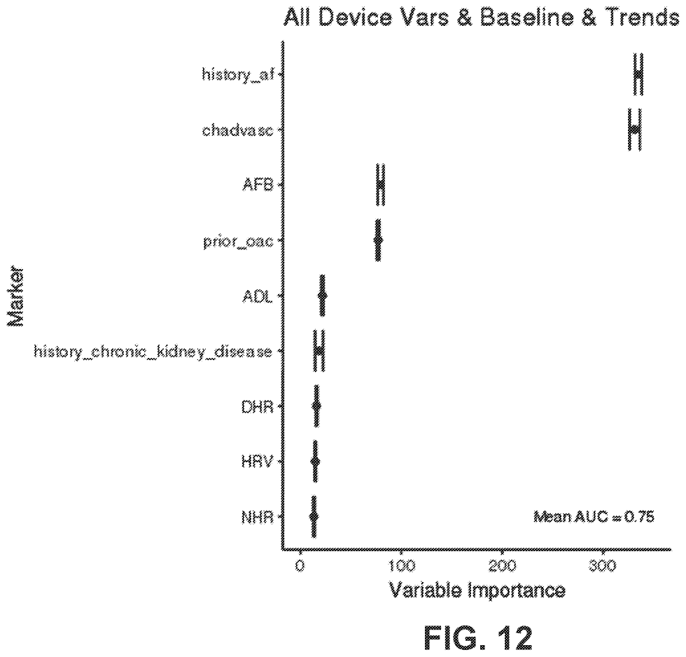

FIG. 12 is another chart illustrating experimentally-determined statistical significances of a plurality of patient parameters in predicting stroke. FIG. 12 is similar to FIG. 11, but includes additional patient parameters. In particular, FIG. 12 includes history of AF, CHADS-VASc score, prior oral anticoagulant (prior_oac), and history of chronic kidney disease. While FIG. 12 illustrates that prior stroke is 13× more significant of predictor of stroke than AF burden, AF burden is the leading predictor after CHADS-VASc.

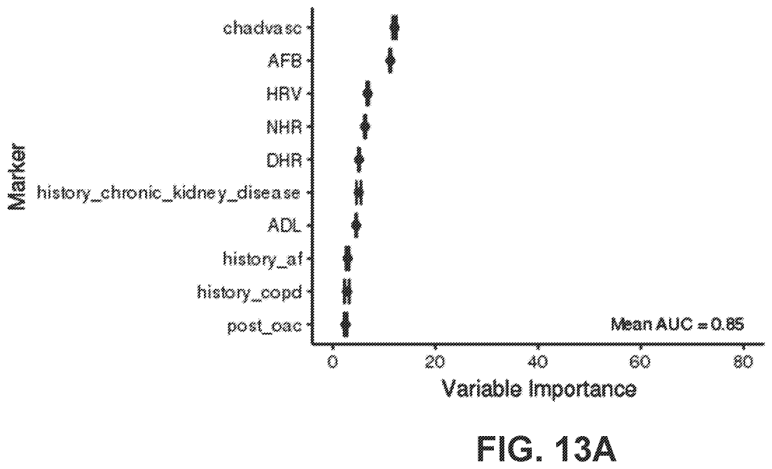

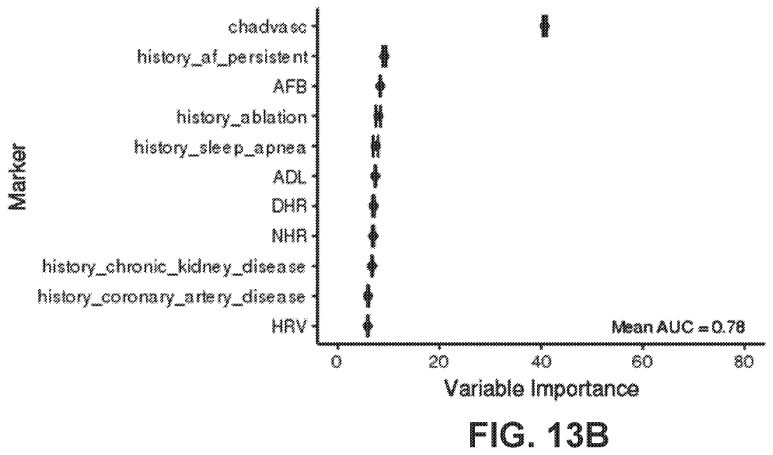

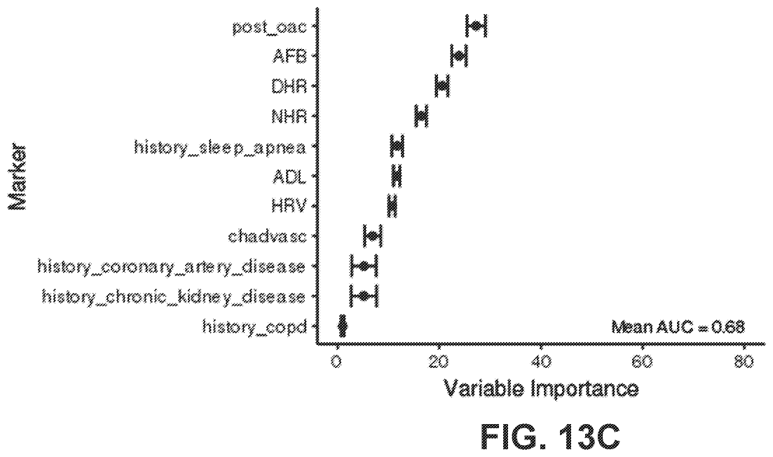

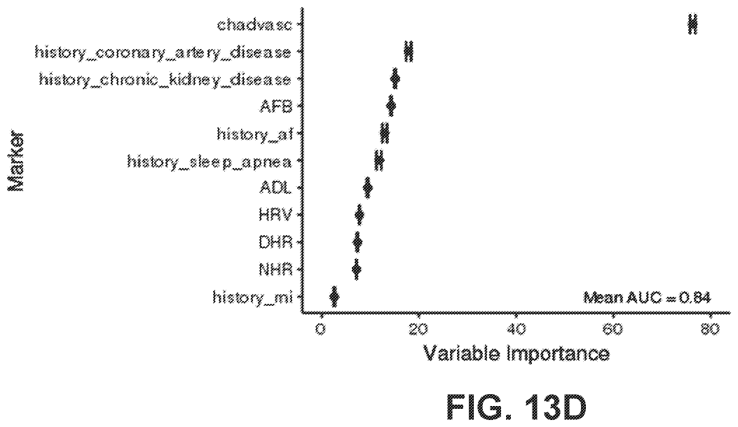

FIGS. 13A-13D are charts illustrating experimentally-determined statistical significances of a plurality of patient parameters in predicting stroke for different patient populations. FIG. 13A illustrates the statistical significances of the plurality of patient parameters in predicting stroke for patients with prior AF ablation. FIG. 13B illustrates the statistical significances of the plurality of patient parameters in predicting stroke for patients with prior AF management. FIG. 13C illustrates the statistical significances of the plurality of patient parameters in predicting stroke for patients with prior stroke. FIG. 13D illustrates the statistical significances of the plurality of patient parameters in predicting stroke for patients in whom AF is suspected by not confirmed.

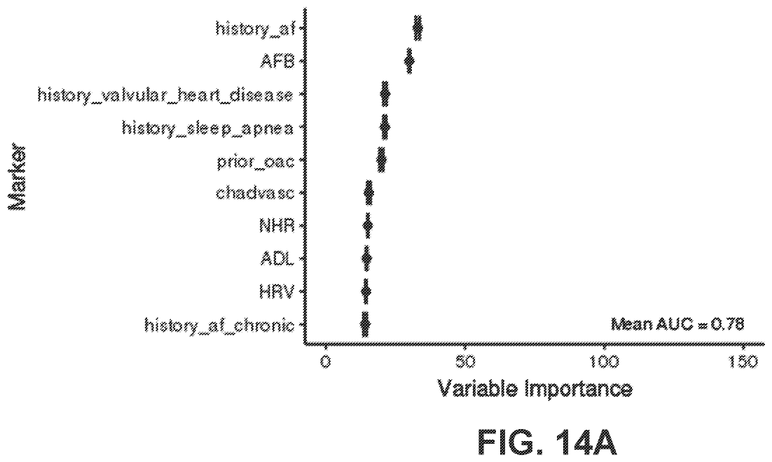

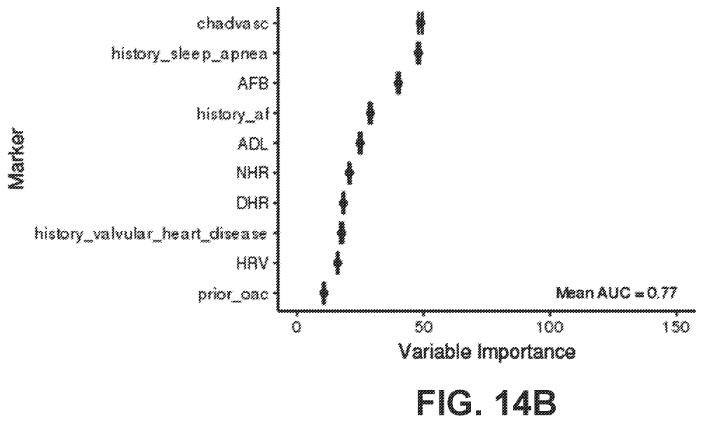

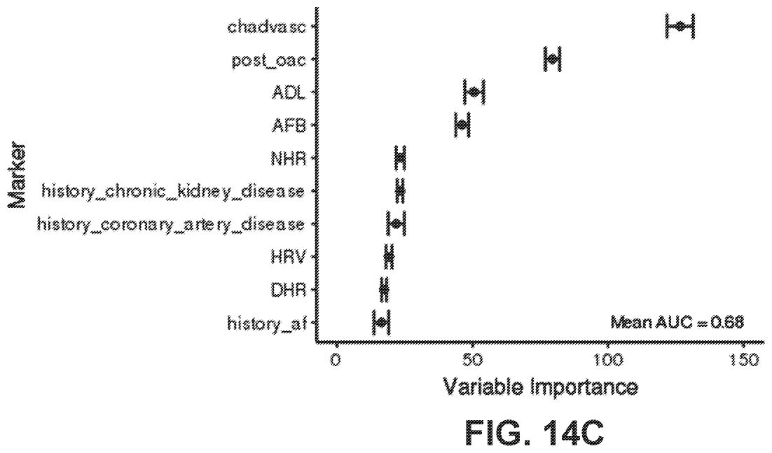

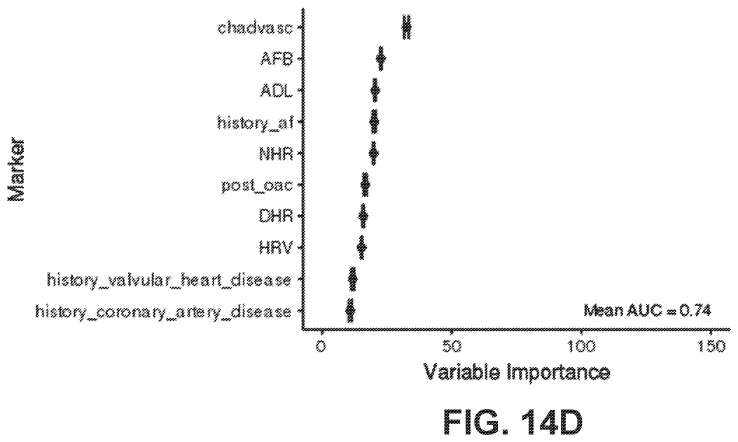

FIGS. 14A-14D are charts illustrating experimentally-determined statistical significances of a plurality of patient parameters in predicting hospitalization (a subset of health care utilization) for different patient populations. FIG. 14A illustrates the statistical significances of the plurality of patient parameters in predicting stroke for patients with prior AF ablation. FIG. 14B illustrates the statistical significances of the plurality of patient parameters in predicting stroke for patients with prior AF management. FIG. 14C illustrates the statistical significances of the plurality of patient parameters in predicting stroke for patients with prior stroke. FIG. 14D illustrates the statistical significances of the plurality of patient parameters in predicting stroke for patients in whom AF is suspected by not confirmed.

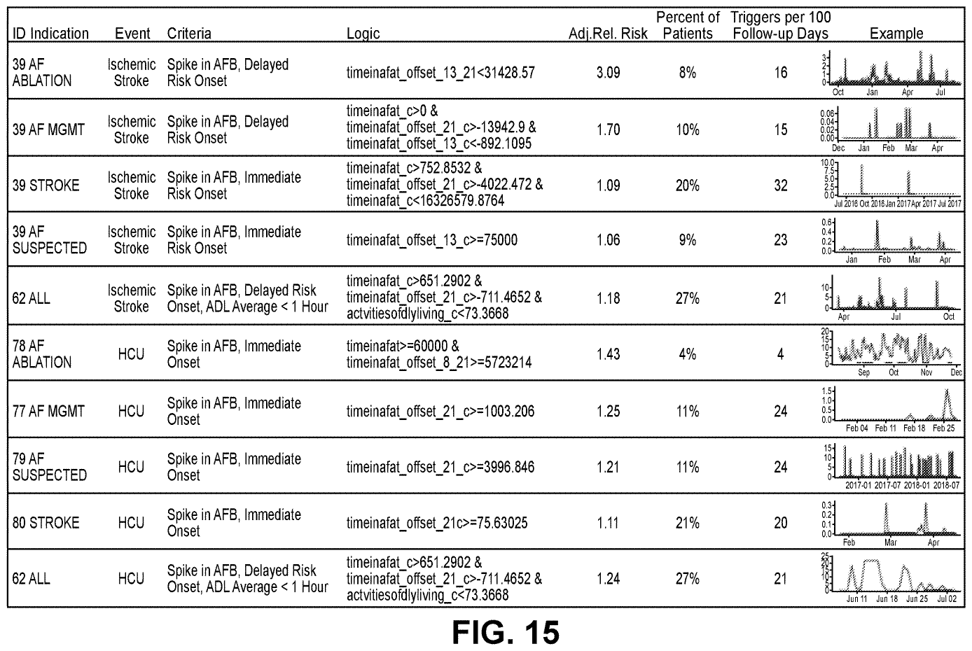

FIG. 15 is a chart illustrating analysis of AF burden pattern features for predicting stroke and health care utilization (HCU). The analysis indicates that for both stroke and HCU, a spike in AF burden, in some cases paired with a low patient activity level, is predictive of the event occurring within a timeframe.

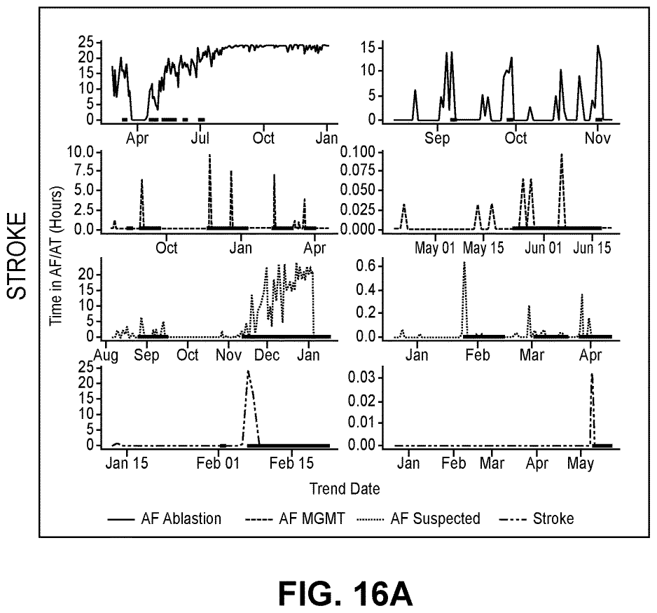

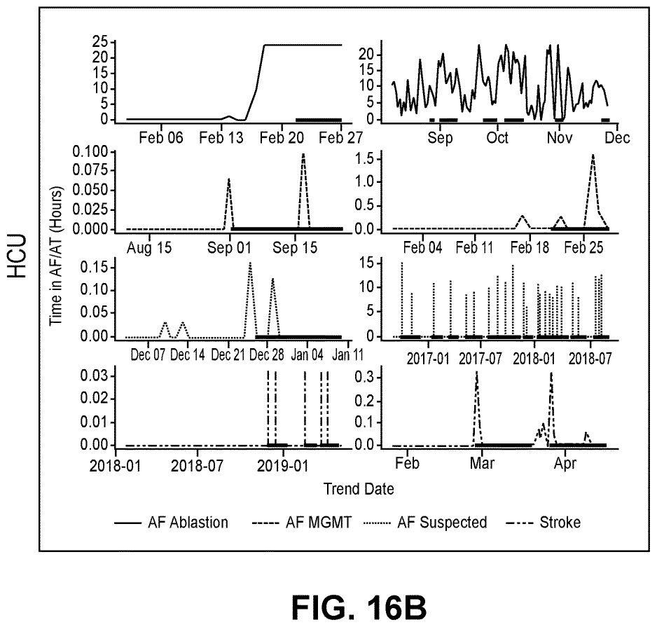

FIGS. 16A and 16B are diagrams illustrating AF burden patterns in patients who experience stroke or a health care utilization event, respectively, for various patient populations. AF burden patterns such as those illustrated in FIGS. 16A and 16B signal subclinical changes that indicate periods of heightened risk for stroke and HCU.

A retrospective cohort study of patients with ICMs, including the Reveal LINQ™ ICM, was performed to determine whether rules-based algorithms that examine change from baseline ICM-based parameters can be used to stratify risk of near-term HCU. The occurrence of HCU as a study end point was obtained from deidentified claims data. A patient was labeled as having an occurrence of HCU if their claims history included at least one encounter from an in—or outpatient hospital, emergency room, or ambulatory surgical center with a cardiovascular DRG or diagnosis code. The first occurrence of HCU was recorded if a patient had multiple utilizations.

ICM-based diagnostic parameters evaluated in the study included daily total AT/AF burden (milliseconds/day), total patient activity, e.g., time with supra-threshold patient motion (minutes/day), average ventricular rate (night and day), and HRV. Patients with less than 21 days of daily follow-up after implant, or with a gap in follow-up greater than or equal to 30 days, were excluded from the cohort. Missing data resulting from a gap in daily follow-up was interpolated by forward-filling the last known value for each diagnostic parameter. Follow-up history was limited to two years unless there was an HCU, in which case follow-up ended the day prior to the event. Patients without any device-detected time in AT/AF within the two-year follow-up period were excluded from the cohort.

To define diagnostic temporal patterns for the study, each parameter, at each patient follow-up date, was evaluated as a cumulative moving average (CMA) from the day after implant and as an SMA of different historical periods (1, 2, 3, 5, 8, 13, and 21 days) starting 21 days after implant. For the study, offset SMA_{a_b} denoted the difference between SMAₐ and SMA_{b} where the longer period SMA is subtracted from the shorter period SMA (i.e., a<b). An offset of period p with its respective CMA was denoted as SMA_{p_c}.

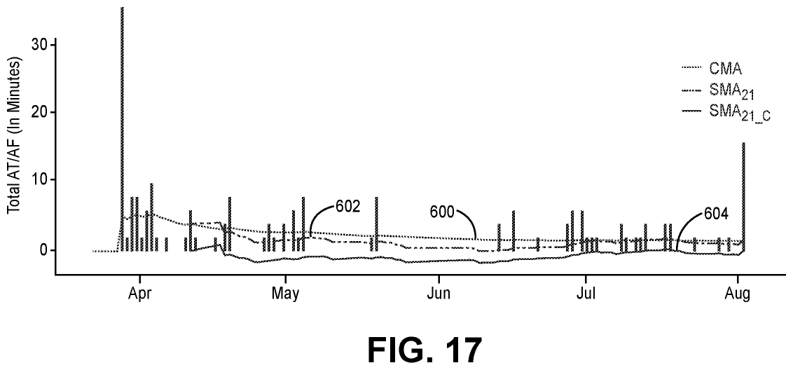

FIG. 17 is a graph illustrating detected AT/AF time (burden) over the course of a monitoring period. The vertical bars illustrate AF burden (AT/AF time) for sub-periods, in this case days, during which the patient experienced AT/AF. The graph of FIG. 17 further includes three trend lines illustrating, respectively, the CMA of AF burden 600, the 21-day SMA of AF burden 602, and the difference between the 21-day SMA and the CMA of AF burden 604.

For the study, the occurrence of HCU was treated as an unbalanced, binary, classification problem. A recursive partitioning & regression tree algorithm (RPART) was used to predict which follow-up days had an occurrence of HCU using diagnostic parameters and moving average offsets as predictors. Random sampling methods for imbalanced learning were used within a bootstrapping routine to promote algorithm convergence and to improve classifier accuracy. HCU events were oversampled by labeling the five days prior to an occurrence as an event. For patients who experienced an HCU, follow-up ended on the day prior to the occurrence to prevent the use of device measurements taken on the same day the event happened, a situation that would introduce look ahead bias into the modeling. For each bootstrap iteration:
1. Patients were randomly partitioned into training (70%) and validation (30%) sets.
2. For the training set, non-labeled days were undersampled to equal the number of labeled days.
3. A classification tree using 10-fold cross validation was fit on the balanced training set.
4. The model fit in Step 3 was pruned to its minimum cross validation error.
5. Split information from the model fit in Step 4 was saved.
6. The unbalanced validation set was classified using the model fit from Step 4.
7. Classification statistics for each terminal node from Step 6 were saved.

Each split for a terminal node was recorded as a 3-tuple, [predictor name, comparison, index], along with its respective HCU rate and patient count for both training and validation sets. Each split was saved as a separate entry if a node had multiple splits. In such a case, the utilization rates and patient counts would be the same for all splits in each node.

A scatterplot of decision tree terminal nodes showing the relationship between labeled HCU rate and the percent of patients was used to identify patterns in the AF burden classification tree structure that would stratify healthcare event risk. The algorithm for defining these patterns was:
1. Visually identify areas in the scatterplot with a local maximum in patient percent.
2. If the area is unique to Time in AT/AF, define rectangular coordinates for event risk and patient percent that enclose the area.
3. If the area is not unique to Time in AT/AF, then
   i. Set the upper and lower boundary for patient percent equal to the local maximum
   ii. Subtract 0.01 from the lower boundary for patient percent.
   iii. Set the lower and upper boundaries for event rate to the respective locations where the scatterplot intersects with the lower patient percent boundary defined in the preceding step.
4. Select nodes within the rectangular area.
5. Group by the pair [predictor name, comparison]
6. Calculate the number of times each pair is selected, the number of times each pair is selected as a percent of all bootstrapped classification trees, and the mean of [index] values.
7. If the area is not unique to Time in AT/AF, repeat Steps 3.ii-6 until the modal predictor is selected in at least 10% of all classification trees.
8. Rank order 3-tuples [predictor, comparison, mean index value] in descending order by selection rate
9. Identify the elbow in selection rate (i.e. where the selection rate drops by approximately 50%).
10. Define an AF burden pattern as the 3-tuples having a selection rate above the elbow point identified in the preceding step.

Descriptive analyses and tests of equal proportions were performed to compare the odds ratio for AF burden patterns with clinically relevant thresholds for duration and quantity.

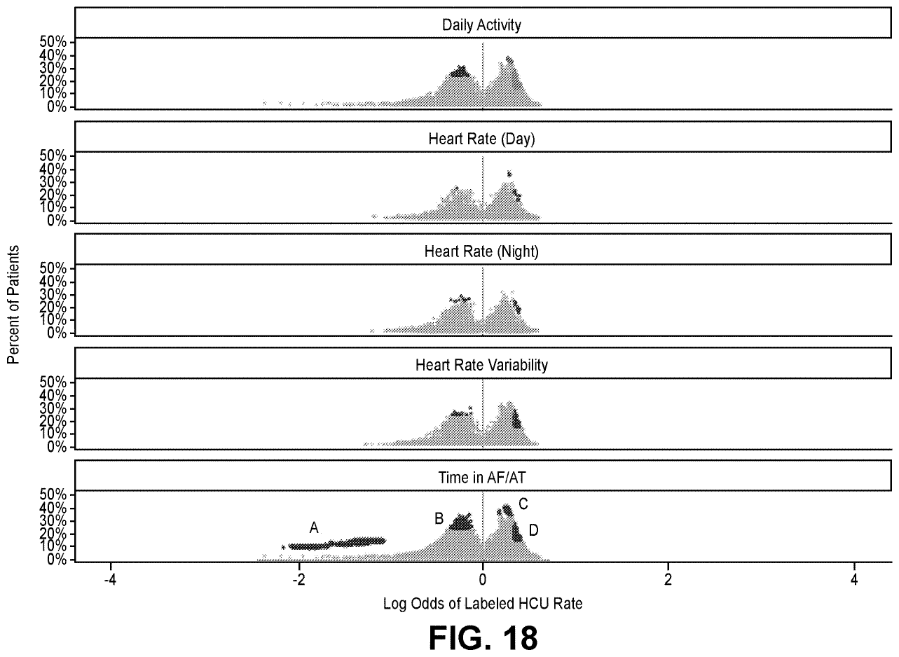

The bootstrapping routine was run 3,000 times to create an equal number of classification trees. FIG. 18 presents a scatterplot of 50,751 terminal nodes by labeled HCU rate and percent of patients for the balanced training data. A point on a plot represents a unique terminal node. A single node can be represented across diagnostic parameters when its definition includes multiples splits with a different parameter for each split (e.g., time in AT/AF>1 hour & daily activity<100 minutes & nighttime heart rate>80 beats per minute). Three local maxima were identified and denoted as shaded areas A, B and C. Missing (A & C) or infrequent (B) nodes for daily activity and heart rate parameters suggest the areas are largely defined by Time in AT/AF. Area D is derived from the analysis of areas A, B & C and is defined later in the results.

TABLE 2
Top splits per terminal node distribution for the training data

| Area | Predictor | Comparison | Mean | Std | Count | Selection |
|---|---|---|---|---|---|---|
| A | timeinafat_{—}c | < | 930 | 193 | 3,000 | 100.00% |
| | timeinafat_offset_3_c | >= | −172 | 56 | 5 | 0.17% |
| | timeinafat_offset_5_c | >= | −168 | 2 | 5 | 0.17% |
| | timeinafat_offset_1_c | >= | −166 | | 1 | 0.03% |
| B | timeinafat_{—}c | >= | 900 | 214 | 454 | 15.13% |
| | timeinafat_{—}offset_{—}21_{—}c | < | −675 | 564 | 399 | 13.30% |
| | timeinafat_{—}offset_{—}1_{—}21 | >= | −75,656 | 182,026 | 371 | 12.37% |
| | timeinafat | < | 1,37 [symbol] ,950 | 2,126,201 | 179 | 5.97% |
| | timeinafat_c | < | 3,869, [symbol] 1 [symbol] | 3,238,723 | 111 | 3.70% |
| C | timeinafat_{—}c | >= | 950 | 342 | 950 | 31.67% |
| | timeinafat_{—}offset_{—}21_{—}c | >= | −721 | 566 | 938 | 31.27% |
| | activitiesofdlyliving_offset_21_c | >= | −11 | 2 | 140 | 4.67% |
| | heartratevariability_offset_21_c | >= | −6 | 1 | 33 | 1.10% |
| | timeinafat | < | 390,000 | 365,861 | 12 | 0.40% |
| D | timeinafat_{—}c | >= | 170,559 | 552,040 | 256 | 8.53% |
| | timeinafat_{—}offset_{—}21_{—}c | >= | −4,771 | 61,172 | 231 | 7.70% |
| | activitiesofdlyliving_{—}c | < | 76 | 8 | 209 | 6.97% |
| | activitiesofdlyliving_offset_21_c | >= | −9 | 4 | 33 | 1.10% |
| | heartratevariability_offset_21_c | >= | −6 | 1 | 32 | 1.07% |

[symbol]

indicates data missing or illegible when filed

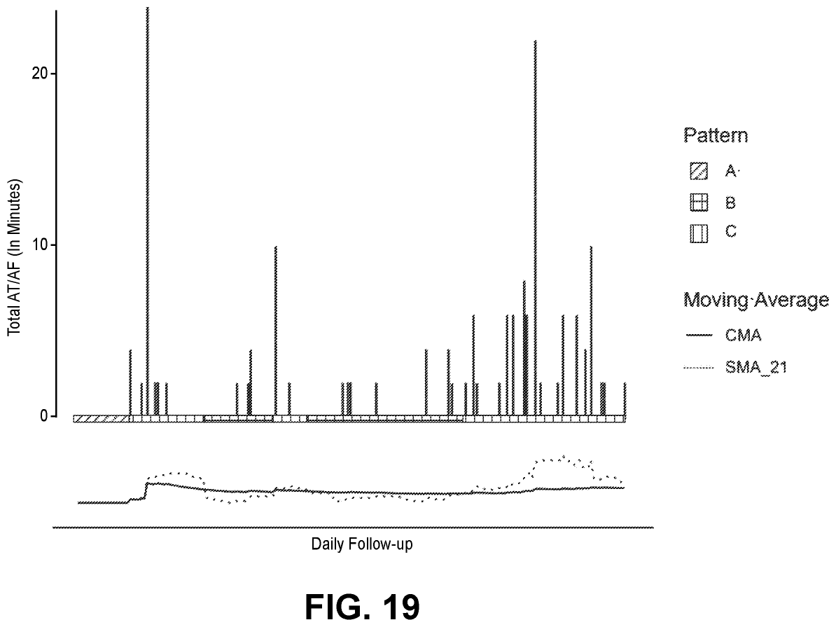

Table 2 (above) presents a summary of the top five splits by area. Splits with a selection rate above their respective elbow point are in bold. Together, these highlighted splits define the AF burden pattern for a given area. Pattern A is defined by an AF burden CMA less than approximately 1 second. The pattern is present in all 3,000 decision trees and describes the follow-up period prior to the first detection of AT/AF (77% of occurrences) and the period of relative sinus rhythm recovery after device detected AT/AF (23% of occurrences). Pattern C is defined by an AF burden CMA greater than approximately 1 second and an AF burden 21-day SMA that is approximately greater than its historical average. The pattern is present in 25% of all decision trees and describes a relative spike or increasing trend in daily AF burden. Pattern B is defined by an AF burden CMA greater than approximately 1 second, but unlike pattern C, it has a decreasing AF burden 21-day SMA that is less than its historical average. The increasing 1-day SMA (daily burden) relative to the 21-day SMA suggests that pattern C signals a period of sporadic, below average burden, relative to the patient, that can occur after a period of elevated burden. FIG. 19 presents an example graphical illustration of these patterns in AF burden data mapped for a single HCU patient.

Labeled HCU rate and patient percent were calculated on the training data for AF burden quantity and duration thresholds. The quantity threshold was defined as daily AF burden greater than 5% (72 minutes); the duration threshold was defined as continuous AF greater than one hour. The log odds event rate was 0.369 and 0.386 and the patient percent was 12.9% and 7.6% for the respective thresholds. Table 2, Area D presents a summary of the top five splits in the terminal node scatterplot where the log odds event rate was greater than 0.369 and the percent of patients was greater than 12.9%. The top three splits define a partial substructure of pattern C where the CMA of daily activity drops below 76 minutes. When applied to a balanced training set, pattern D is selected 10.4% of the time and has a log odds ratio of 0.368, a value that is not statistically different from the other log odds ratios (Poisson regression, p>0.5 for all threshold coefficients).

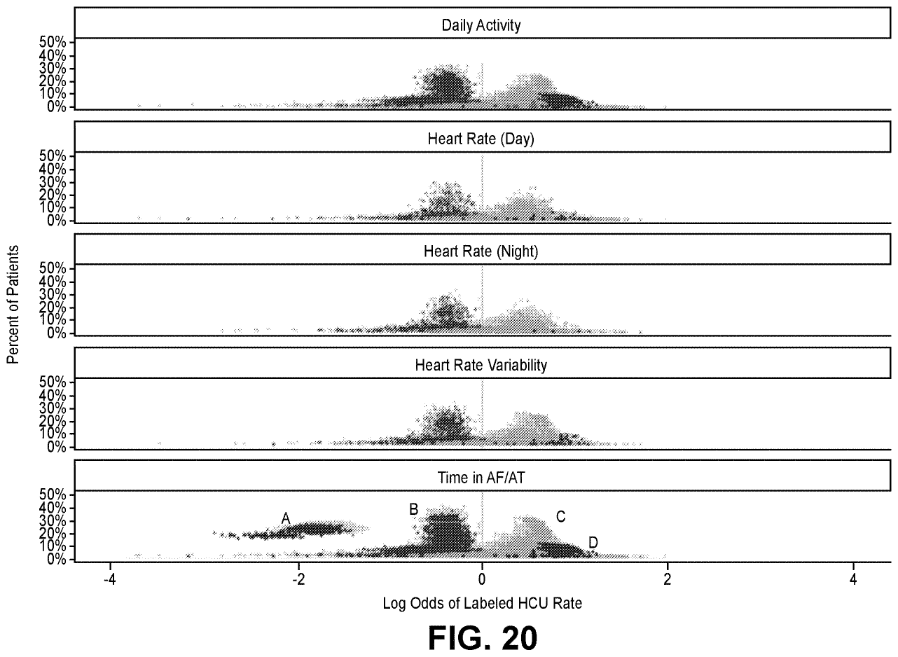

FIG. 20 presents a scatterplot of scored terminal nodes by labelled HCU rate and patient percent for the unbalanced validation set. The overall distribution is similar in shape to the training data (FIG. 18). Different shades of color show different threshold patterns with the lightest shaded points representing nodes not covered by a pattern. AF burden patterns (A-C) provide broad coverage of the terminal node distribution with clear segmentation of event risk (B versus A, odds ratio (OR) 3.82, 95% CI 3.59-4.07; C versus A, OR 8.25, 95% CI 7.84-8.69). Including a daily activity threshold (D) provides more specific coverage with the greatest risk for HCU among AF burden patterns (D versus A, OR 11.66, 95% CI 10.63-12.79).

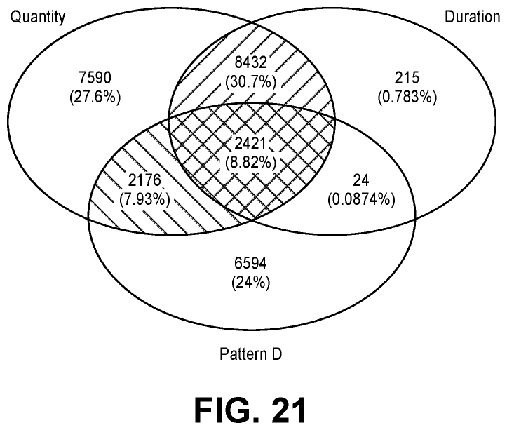

FIG. 21 presents a Venn diagram of AF burden threshold counts for the validation set. Table 3 (below) presents statistics for each threshold and their mutually exclusive subsets. Approximately 32% (6,594/20,858) of pattern D thresholds are mutually exclusive to quantity and duration thresholds and represent a 33% (212/644) increase in event capture rate. A test for odds ratio differences using Poisson regression showed a statistically significant coefficient for the intersection of all three thresholds (p<0.10); the remaining coefficients were not statistically different (p>0.10 for all coefficients). Approximately 23% of patients experienced just the pattern D threshold at an expected rate of 18.2% of follow-ups, or 66 days per year.

TABLE 3
AF burden threshold statistics for the validation data

| AF Burden Threshold | Count | Events | Odda | [95% C.I.] | Patients | Follow-ups |
|---|---|---|---|---|---|---|
| Sets | | | | | | |
| Quantity | 20,619 | 840 | 1.93 | [1.79 - 2.08] | 59.7% | 18.1% |
| Duration | 11,092 | 390 | 2.19 | [1.98 - 2.41] | 52.6% | 10.8% |
| Pattern D | 11,215 | 439 | 2.43 | [2.22 - 2.67] | 28.2% | 28.7% |
| Mutually Exclusive Subsets | | | | | | |
| Duration | 218 | 2 | 0.58 | [0.10 - 2.29] | 12.0% | 0.6% |
| Quantity | 7,590 | 163 | 1.34 | [1.14 - 1.50] | 35.1% | 10.3% |
| Quantity & Daration | 8,432 | 262 | 1.86 | [1.64 - 2.10] | 41.9% | 9.2% |
| Pattern D | 6,594 | 212 | 2.00 | [1.78 - 2.20] | 23.3% | 18.2% |
| Pattern D & Quantity | 2,176 | 91 | 2.60 | [2.11 - 3.19] | 16.1% | 7.8% |
| Pattern D & Quantity & Duration | 2,421 | 134 | 3.44 | [2.91 - 4.07] | 17.8% | 8.4% |
| Pattern D & Duratkm | 24 | 2 | 5.18 | [0.91 - 17.70] | 2.4% | 0.3% |

In Table 3, Count indicates the number of times the threshold was met; Events, the number of labeled HCUs; Odds, the ratio of event count to threshold count divided by the group mean event rate for the validation set; Patients, the number of patients with at least one day meeting the threshold as a percent of total patients in the validation set; Follow-ups, the number of days meeting the threshold as a percent of total follow-up days for patients with at least one occurrence of the threshold. Note: counts are mutually exclusive per follow-up days, not by patient. A patient may experience different thresholds across follow-up days. Therefore, patient and follow-up percentages will not sum to 100%.

The analysis of AF burden patterns in the study confirm the correlation between increased burden and risk and, more particularly, that a growing trend in AF burden (e.g., daily) over time is associated with a greater risk for HCU, especially when accompanied with a decline in daily activity. Patterns for AF burden amounts less than approximately 1-hour predict healthcare events on par with quantity and duration thresholds greater than 1-hour. AF burden patterns proved additional event capture that complements quantity and duration thresholds. AF burden as a risk factor for HCU is relative to a patient's historical burden.

The study illustrates the value of AF burden and patient activity as parametric data from which features may be derived and then applied to an algorithm or model to determine a likelihood of an event, such as an HCU event, as described herein. The features derived from AF burden may include AF burden pattern features, such as a change, e.g., spike or increase, in AF burden relative to an overall AF burden trend. For example, AF burden pattern features may include one or more offsets between SMAs for different look-back periods and/or between an SMA for a look-back period and a CMA. As described herein, the model to which such features are applied may be machine learned or rules-based, e.g., involving decision trees and/or thresholds.

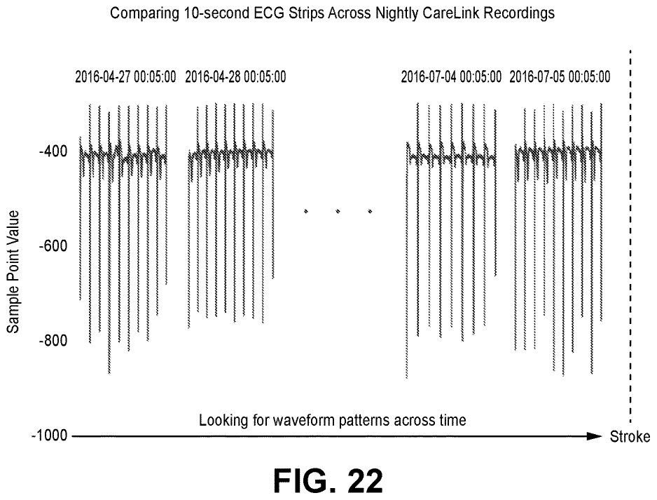

Monitoring system 222 may determine a risk level of a health event based on changes, including sometimes subtle changes, in ECG data, such as changes in sinus rhythm, over a period of time. In some examples, monitoring system 222 may receive ECG data of patient 4. FIG. 22 illustrates an example in which monitoring system 222 receives strips of ECG data of patient 4. In the example shown in FIG. 22, the strips of ECG data are 10-seconds in length and were sampled nightly from April 27^{th} to July 5^{th}. In some examples, the length of the ECG strip recording or the number of heartbeat intervals may vary. In addition, the time of day for each ECG recording and the number of days used for ECG data comparison may vary as well.

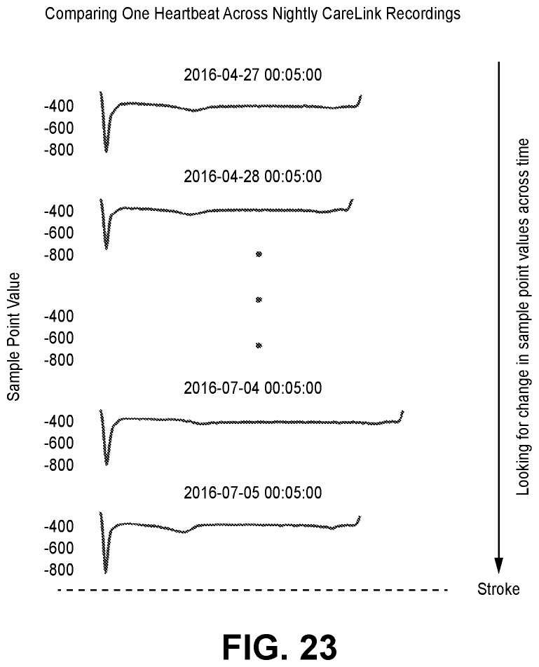

FIG. 23 illustrates heartbeat intervals sampled from the ECG data strips shown in FIG. 22. In the example shown in FIG. 23, the heartbeat intervals correspond to an RR-interval. In some examples, monitoring system 222 may sample intervals from a strip of ECG data that correspond to one heartbeat or summarized intervals from a strip of ECG data into a single heartbeat interval. As shown in FIG. 22, the sampled heartbeat intervals over the period of days are arranged on the graph to be compared. In some examples, monitoring system 222 may take each sampled or summarized interval at the same time of day so that the sampled intervals may be compared. For example, FIG. 23 shows each sampled interval being taken at 00:05:00 for each respective day. In some examples, the sampled or summarized intervals may be taken when a patient is at rest, such as at nighttime, to have more consistent patient variables, such as exercise, circadian patterns, etc. In some examples, each obtained heartbeat interval from the ECG data may be a single heartbeat interval or an average of a plurality of adjacent and/or neighboring heartbeats from the ECG data, such as, for example, an average of 10 adjacent or neighboring heartbeats.

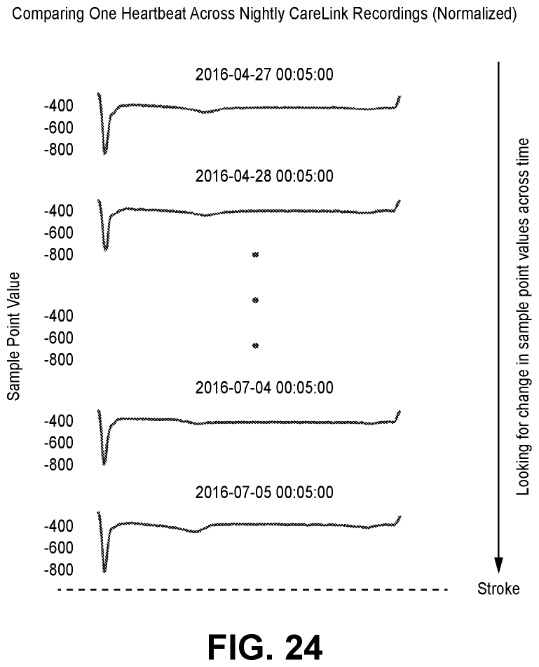

As shown in FIG. 23, sampled or summarized heartbeat intervals may have different interval lengths for a variety of reasons, such as due to a patient having a varying heart rate on different days. FIG. 24 illustrates monitoring system 222 normalizing the respective sampled heartbeat intervals shown in FIG. 23 to have a same interval length to be compared. Upon the sampled heartbeat intervals being normalized, monitoring system 222 may sample each respective heartbeat interval at a number of points, such as 400 points, so monitoring system 222 may compare each point in a normalized heartbeat interval to a corresponding respective point over time. For example, monitoring system 222 may compare point 100 for the sampled heartbeat intervals of April 27^{th} to July 5^{th} to each other to track bow the value of point 100 changes over time. In the example shown in FIGS. 23-24, 400 sample points were taken for each sampled interval and normalized to be compared. In some examples, the number of sample points may be greater than 400 or less than 400. In some examples, the number of sample points may be between 200 and 2000 sample points per sample interval, such as 200, 300, 400, 500, 600, 700, 800, 900 or 1000 sample points per sampled interval. In some examples, the number of samples points may be other amounts as well.

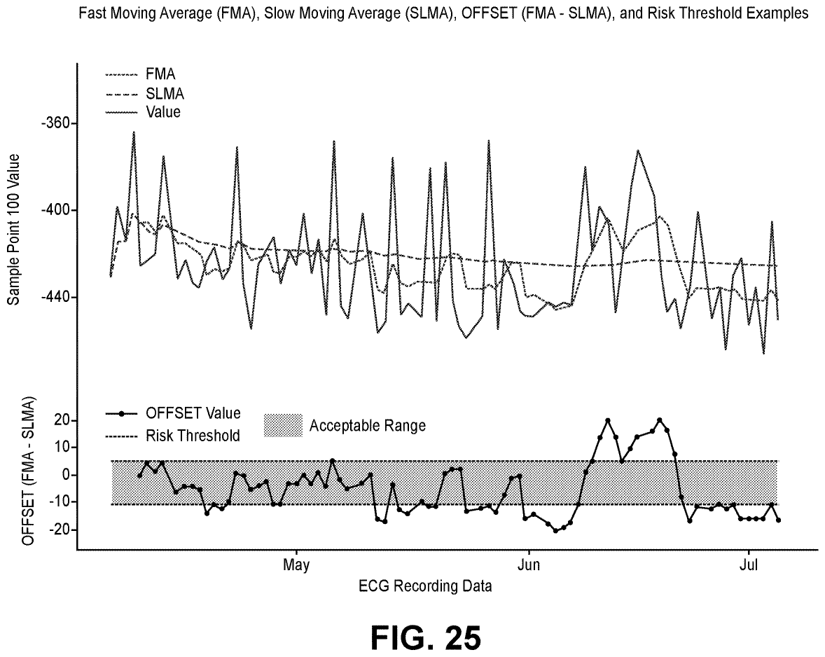

FIG. 25 illustrates an example of monitoring system 222 determining a value of a fast-moving average (FMA) of sample point 100, a slow-moving average (SLMA) of sample point 100, and a value of each sample point itself of sample point 100, of the sampled intervals shown in FIG. 24, over a period of time (e.g., from April 27^{th} to July 5^{th}). In some examples, monitoring system 222 summarizes a trend with at least the FMA and SLMA and uses a comparison or offset of the FMA and SLMA to capture a clinically significant change in the patient parameter. The FMA is a shorter period moving average than the SLMA. For example, the longer term SLMA includes less recent values of the patient parameter than the shorter period FMA. In some examples, a period of time for the SLMA may be 20 days while the period of time for the FMA may be 8 days. In some examples, a period of time for the SLMA may be 25 days while the period of time for the FMA may be 10 days. The period of time for the SLMA and the FMA may be a variety of different amounts of days, but the period of time for the SLMA is greater than the period of time for the FMA. In some examples, the period of time for the SLMA may be 10 days more than the period of time for the FMA. In other examples, the period of time for the SLMA may be 8, 12, 15, 17, 20, 30, or 50 days more than the period of time for the FMA, The more recent days may be included in both the SLMA and FMA.

FIG. 25 further illustrates monitoring system 222 determining a difference (labeled as OFFSET value in FIG. 25) between the FMA and the SLMA of sample point 100 for the sampled intervals over a period of time (e.g., from April 27^{th} to July 5^{th}). FIG. 25 further shows a risk threshold. A risk threshold may be determined by the variation of FMA and SLMA values over a period of time as well as the difference in their respective values over a period of time. A risk threshold may also be specified as an absolute value. Monitoring system 222 may determine a risk of a health event based on a difference between the FMA and SLMA falling outside of the acceptable range, as shown in FIG. 25. In some examples, the health event may be a cardiovascular event. The health event may be a stroke. In some examples, the health event is a health care utilization event, such as a hospitalization. In some examples, the health event may be sudden cardiac arrest (SCA). In some examples, the health event may be a myocardial infarction (MI).

In addition, an amount the determined difference (e.g., OFFSET) between the FMA and the SLMA falls outside the acceptable range may also indicate a risk of a health event. In some examples, a number of consecutive sample intervals having the determined difference (e.g., OFFSET) occurring outside the acceptable range may indicate a risk of a health event. In addition, a number and/or percentage of sample intervals having the determined difference (e.g., OFFSET) occurring outside the acceptable range over a period of intervals may indicate a risk of a health event. Also, the maximum or minimum difference (e.g., OFFSET) may indicate a risk of a health event.

In some examples, monitoring system 222 may calculate an absolute value of the determined difference (e.g., OFFSET) between the FMA and the SLMA and compare the absolute value of the determined difference to a risk threshold value. Monitoring system 222 may determine a risk of a health event based on the comparison of the absolute value of the determined difference (e.g., OFFSET) between the FMA and the SLMA to the risk threshold value. For example, the absolute value of the determined difference (e.g., OFFSET) between the FMA and the SLMA being greater than the risk threshold may indicate a risk of health event, such as stroke. In some examples, an amount the absolute value of the determined difference (e.g., OFFSET) between the FMA and the SLMA is greater than the risk threshold may also indicate a risk of a health event. In some examples, a number of consecutive sample intervals having the absolute value of the determined difference (e.g., OFFSET) being greater than or equal to a risk threshold may indicate a risk of a health event. In addition, a number and/or percentage of sample intervals having the absolute value of the determined difference (e.g., OFFSET) being greater than or equal to a risk threshold over a period of intervals may indicate a risk of a health event. Also, the maximum or minimum difference (e.g., OFFSET) over a period of intervals may indicate a risk of a health event. In some examples, monitoring system 222 may flag one or more sample intervals when the comparison of the absolute value of the determined difference (e.g., OFFSET) between the FMA and the SLMA to the risk threshold value indicates a risk of a health event, such as discussed above. For example, monitoring system 222 may flag a sample interval, for example June 15^{th}, when the absolute value of the determined difference (e.g., OFFSET) between the FMA and the SLMA is greater than the risk threshold.

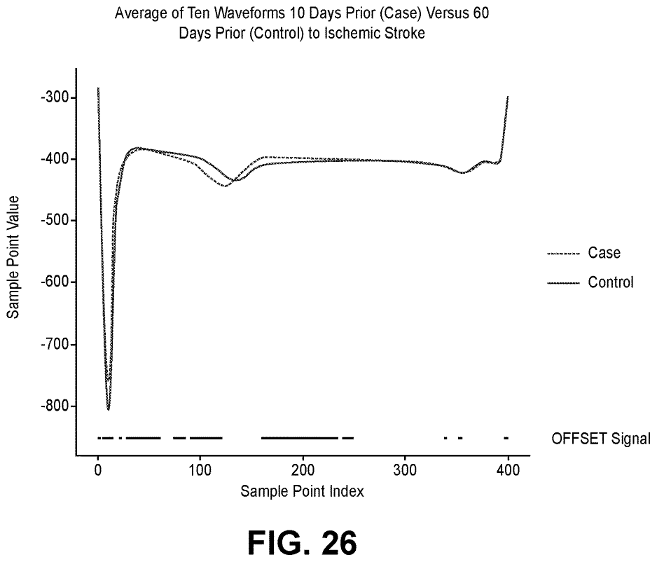

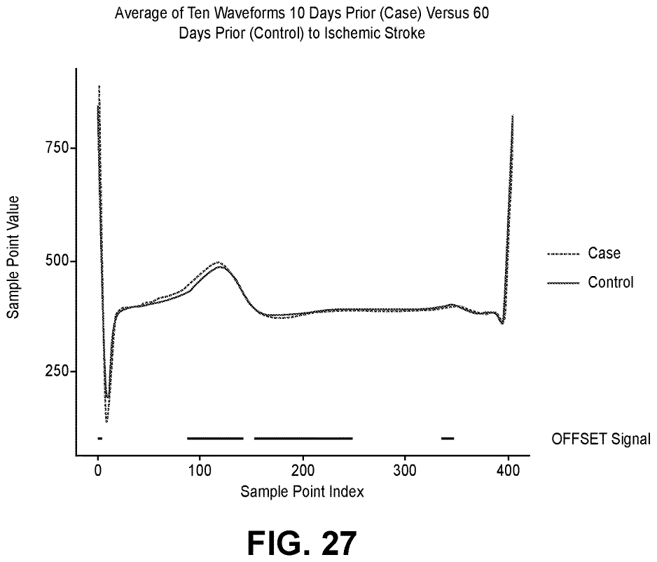

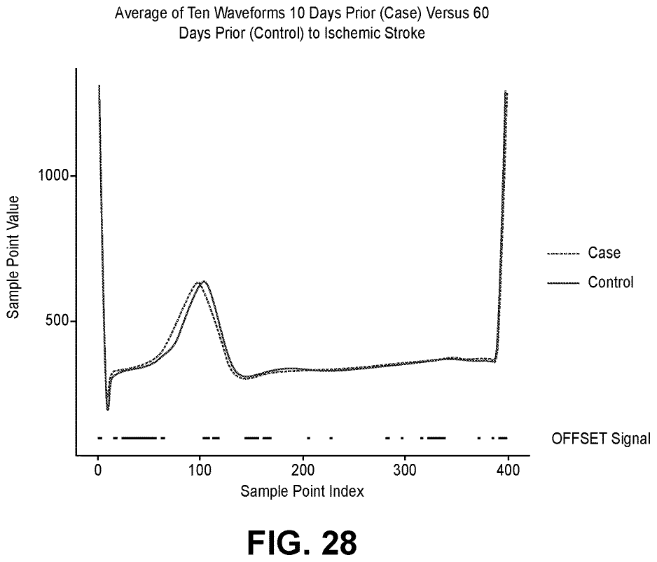

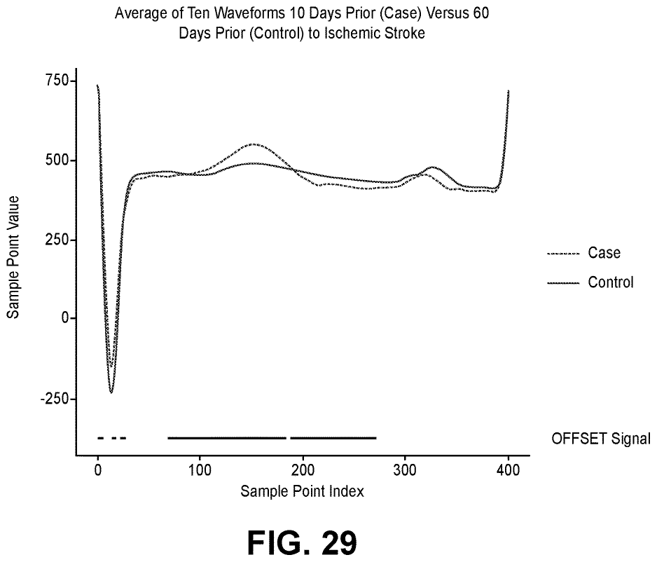

In some examples, when monitoring system 222 flags one or more sample intervals, monitoring system 222 may display a graph showing an average of sample point parameters for different period lengths over time. In addition, monitoring system 222 may also display an indication of what parameter points had the determined difference (e.g., OFFSET) between the FMA and the SLMA being greater than the risk threshold (e.g., OFFSET signal). FIGS. 26-29 show examples for the 10 days prior (e.g., case average) to the flagged interval date compared to a 10-day average (e.g., control average) that was taken 60 days prior to the case average of flagged intervals being displayed as described above. In some examples, monitoring system 222 may display such graphs to a clinician, service provider, or other treatment decision maker to determine a course of treatment in response to the displayed graphical data.

In some examples, monitoring system 222 may receive electrocardiogram (ECG) data of a patient, wherein the ECG data is generated by one or more sensing devices of the patient based on physiological signals of the patient sensed by the one or more sensing devices. In some examples, monitoring system 222 obtain a plurality of heartbeat data from the ECG data. In some examples, monitoring system 222 may determine a slow-moving metric based on the heartbeat data during a first period of time and determine a fast-moving metric based on the heartbeat data during a second period of time, the second period of time being less than the first period of time. In some examples, the metric of the slow-moving metric and/or fast-moving metric may include one or more of an average, mode, mean, median, counts, and/or set of values of the heartbeat data over a period of time. For example, monitoring system 222 may determine a slow-moving mode and a fast-moving mode of heartbeat data. In some examples, monitoring system 222 may determine a risk level of a health event for the patient based on the slow-moving metric and the fast-moving metric.

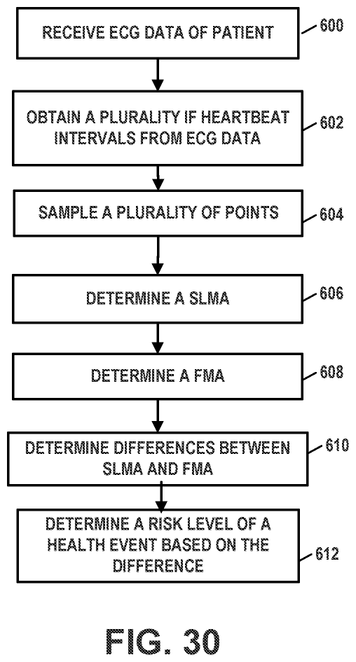

According to the example illustrated by FIG. 30, monitoring system 222 receives ECG data of patient 4 (600). As discussed above, the ECG data may be generated by one or more sensing devices of the patient based on physiological signals of the patient sensed by the one or more sensing devices. Monitoring system 222 obtains a plurality of heartbeat intervals from the ECG data (602). Monitoring system 222 samples a plurality of points for each respective heartbeat interval of the plurality of intervals (604). As discussed above, in some examples, each obtained heartbeat interval from the ECG data may be a single heartbeat interval or an average of a plurality of adjacent and/or neighboring heartbeats from the ECG data, such as, for example, an average of 10 adjacent or neighboring heartbeats. As discussed above, monitoring system 222 may normalize the plurality of sampled points for each respective heartbeat interval of the plurality of intervals.

Monitoring system 222 determines a slow-moving average for each sample point parameter of respective heartbeat intervals for one period of time (606). Monitoring system 222 determines a fast-moving average for each sample point parameter of respective heartbeat intervals for a second period of time (608). As discussed above, the second period of time is less than the first period of time. Monitoring system 222 determines a difference between the slow-moving average and the fast-moving average for each sample point parameter (610). Monitoring system 222 determines a risk level of a health event for the patient based on the determined differences (612).

As discussed above, monitoring system 222 may determine a comparison value for each sample point parameter by comparing an absolute value of the difference between the slow-moving average and the fast-moving average for each sample point parameter to a difference threshold and determine the risk level of the health event for the patient based on the comparison values for each sample point parameter. In some examples, monitoring system 222 may determine the risk level of the health event for the patient based on a number of consecutive parameter points having the respective comparison value above or equal to the comparison threshold. In some examples, monitoring system 222 may determine the risk level of the health event for the patient based on an amount of the comparison value.

In some examples, monitoring system 222 may generate an output to indicate the risk level of the health event. In some examples, monitoring system 222 may generate suggested responses, such as treatment, changes in medication, suggestion to go to the hospital, to be output in response to determining the risk level of the health event. Accordingly, in some examples, due to monitoring system 222 determining the risk level of the health event for the patient, serious health events, such as stoke, MI, or SCA, may be treated before the health event occurs, which may provide greater care for a patient and/or reduce healthcare costs.

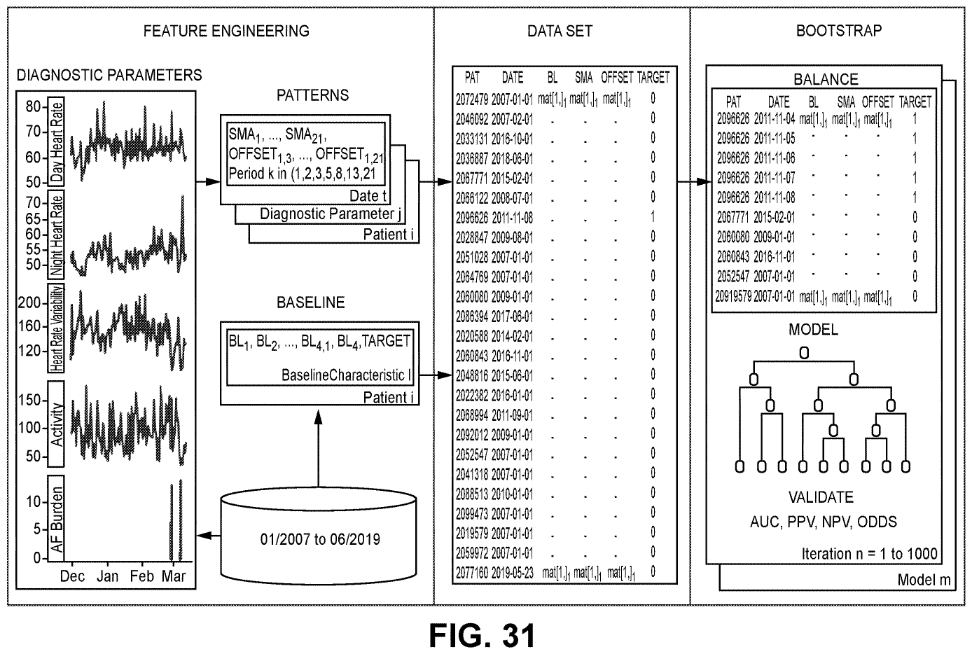

FIG. 31 illustrates an example methodology for rare event prediction using continuous monitoring data. In the example shown in FIG. 31, diagnostic parameter data was partitioned to 70% training and 30% testing, under/oversampled, model fitted, and model tested for each of the 1000 bootstraps. This was repeated for seven different model structures. The bootstrapped mean area under the curve (AUC) and classification statistics were used to measure how well a methodology could predict the occurrence of ischemic stroke.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts of events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes a memory; and processing circuitry coupled to the memory and configured to: receive electrocardiogram (ECG) data of a patient, wherein the ECG data is generated by one or more sensing devices of the patient based on physiological signals of the patient sensed by the one or more sensing devices; obtain a plurality of heartbeat intervals from the ECG data; sample a plurality of points for each respective heartbeat interval of the plurality of intervals; determine a slow-moving average for each sample point parameter of respective heartbeat intervals during a first period of time; determine a fast-moving average for each sample point parameter of respective heartbeat intervals during a second period of time, the second period of time being less than the first period of time; determine a difference between the slow-moving average and the fast-moving average for each sample point parameter; and determine a risk level of a health event for the patient based on the determined differences.

Example 2: The system of example 1, wherein the processing circuitry is configured to obtain the plurality of heartbeat intervals at a respective particular time of day over a plurality of days.

Example 3: The system of any of examples 1 to 2, wherein the processing circuitry is further configured to normalize the plurality of sampled points for each respective heartbeat interval of the plurality of intervals.

Example 4: The system of any of examples 1 to 3, wherein the processing circuitry is further configured to: determine a comparison value for each sample point parameter by comparing an absolute value of the difference between the slow-moving average and the fast-moving average for each sample point parameter to a risk threshold; and determine the risk level of the health event for the patient based on the comparison values for each sample point parameter.

Example 5: The system of any of examples 1 to 4, wherein the processing circuitry is further configured to determine the risk level of the health event for the patient based on a number of consecutive parameter points having the respective comparison value above or equal to the comparison threshold.

Example 6: The system of any of examples 1 to 4, wherein the processing circuitry is further configured to determine the risk level of the health event for the patient based on an amount of the comparison value.

Example 7: The system of any of examples 1 to 6, wherein the health event comprises stroke.

Example 8: The system of any of examples 1 to 6, wherein the health event comprises a health care utilization event.

Example 9: The system of any of examples 1 to 6, wherein the health event comprises sudden cardiac arrest.

Example 10: The system of any of examples 1 to 9, wherein, to determine the risk level of the health event, the processing circuitry is configured to determine a probability of an occurrence of the health event.

Example 11: The system of any of examples 1 to 10, wherein the risk level comprises a risk that the health event will occur within a predetermined time period.

Example 12: The system of any of examples 1 to 11, wherein the processing circuitry is configured to determine whether the risk level of the health event satisfies a criterion.

Example 13: A method includes receiving electrocardiogram (ECG) data of a patient, wherein the ECG data is generated by one or more sensing devices of the patient based on physiological signals of the patient sensed by the one or more sensing devices; obtaining a plurality of heartbeat intervals from the ECG data; sampling a plurality of points for each respective heartbeat interval of the plurality of intervals; determining a slow-moving average for each sample point parameter of respective heartbeat intervals during a first period of time; determining a fast-moving average for each sample point parameter of respective heartbeat intervals during a second period of time, the second period of time being less than the first period of time; determining a difference between the slow-moving average and the fast-moving average for each sample point parameter; and determining a risk level of a health event for the patient based on the determined differences.

Example 14: The method of example 13, wherein the plurality of heartbeat intervals are obtained at a respective particular time of day over a plurality of days.

Example 15: The method of any of examples 13 to 14, wherein the method further comprises normalizing the plurality of sampled points for each respective heartbeat interval of the plurality of intervals.

Example 16: The method of any of examples 13 to 15, wherein the method further comprises: determining a comparison value for each sample point parameter by comparing an absolute value of the difference between the slow-moving average and the fast-moving average for each sample point parameter to a risk threshold; and determining the risk level of the health event for the patient based on the comparison values for each sample point parameter.

Example 17: The method of any of examples 13 to 16, wherein the method further comprises determining the risk level of the health event for the patient based on a number of consecutive parameter points having the respective comparison value above or equal to the comparison threshold.

Example 18: The method of any of examples 13 to 16, wherein the method further comprises determining the risk level of the health event for the patient based on an amount of the comparison value.

Example 19: The method of any of examples 13 to 18, wherein the health event comprises stroke.

Example 20: The method of any of examples 13 to 18, wherein the health event comprises a health care utilization event.

Example 21: The method of any of examples 13 to 18, wherein the health event comprises sudden cardiac arrest.

Example 22: The method of any of examples 13 to 21, wherein determining the risk level of the health event comprises determining a probability of an occurrence of the health event.

Example 23: The method of any of examples 13 to 22, wherein the risk level comprises a risk that the health event will occur within a predetermined time period.

Example 24: The method of any of examples 13 to 23, further comprising determining whether the risk level of the health event satisfies a criterion.

Example 25: A method comprising any combination of the methods of examples 13 to 24.

Example 26: A computer-readable medium storing instructions that when executed by one or more processors cause the one or more processors to perform the method of any of examples 13-24.

Example 27: A system includes a memory; and processing circuitry coupled to the memory and configured to: receive electrocardiogram (ECG) data of a patient, wherein the ECG data is generated by one or more sensing devices of the patient based on physiological signals of the patient sensed by the one or more sensing devices; obtain a plurality of heartbeat data from the ECG data; determine a slow-moving metric based on the heartbeat data during a first period of time; determine a fast-moving metric based on the heartbeat data during a second period of time, the second period of time being less than the first period of time; and determine a risk level of a health event for the patient based on the slow-moving metric and the fast-moving metric.

Various examples have been described. These and other examples are within the scope of the following claims.

1. A system comprising:
a memory; and
processing circuitry coupled to the memory and configured to:
receive electrocardiogram (ECG) data of a patient, wherein the ECG data is generated by one or more sensing devices of the patient based on physiological signals of the patient sensed by the one or more sensing devices;
obtain a plurality of heartbeat intervals from the ECG data;
sample a plurality of points for each respective heartbeat interval of the plurality of intervals;
determine a slow-moving average for each sample point parameter of respective heartbeat intervals during a first period of time;
determine a fast-moving average for each sample point parameter of respective heartbeat intervals during a second period of time, the second period of time being less than the first period of time;
determine a difference between the slow-moving average and the fast-moving average for each sample point parameter; and
determine a risk level of a health event for the patient based on the determined differences.

2. The system of claim 1, wherein the processing circuitry is configured to obtain the plurality of heartbeat intervals at a respective particular time of day over a plurality of days.

3. The system of claim 1, wherein the processing circuitry is further configured to normalize the plurality of sampled points for each respective heartbeat interval of the plurality of intervals.

4. The system of claim 1, wherein the processing circuitry is further configured to:
determine a comparison value for each sample point parameter by comparing an absolute value of the difference between the slow-moving average and the fast-moving average for each sample point parameter to a risk threshold; and
determine the risk level of the health event for the patient based on the comparison values for each sample point parameter.

5. The system of claim 4, wherein the processing circuitry is further configured to determine the risk level of the health event for the patient based on a number of consecutive parameter points having the respective comparison value above or equal to the comparison threshold.

6. The system of claim 4, wherein the processing circuitry is further configured to determine the risk level of the health event for the patient based on an amount of the comparison value.

7. The system of claim 1, wherein the health event comprises stroke.

8. The system of claim 1, wherein the health event comprises a health care utilization event.

9. The system of claim 1, wherein the health event comprises sudden cardiac arrest.

10. The system of claim 1, wherein, to determine the risk level of the health event, the processing circuitry is configured to determine a probability of an occurrence of the health event.

11. The system of claim 1, wherein the risk level comprises a risk that the health event will occur within a predetermined time period.

12. The system of claim 1, wherein the processing circuitry is configured to determine whether the risk level of the health event satisfies a criterion.

13. A method comprising:
receiving electrocardiogram (ECG) data of a patient, wherein the ECG data is generated by one or more sensing devices of the patient based on physiological signals of the patient sensed by the one or more sensing devices;
obtaining a plurality of heartbeat intervals from the ECG data;
sampling a plurality of points for each respective heartbeat interval of the plurality of intervals;
determining a slow-moving average for each sample point parameter of respective heartbeat intervals during a first period of time;
determining a fast-moving average for each sample point parameter of respective heartbeat intervals during a second period of time, the second period of time being less than the first period of time;
determining a difference between the slow-moving average and the fast-moving average for each sample point parameter; and
determining a risk level of a health event for the patient based on the determined differences.

14. A computer-readable medium storing instructions that when executed by one or more processors cause the one or more processors to perform the method of claim 13.

15. A system comprising:
a memory; and
processing circuitry coupled to the memory and configured to:
receive electrocardiogram (ECG) data of a patient, wherein the ECG data is generated by one or more sensing devices of the patient based on physiological signals of the patient sensed by the one or more sensing devices;
obtain a plurality of heartbeat data from the ECG data;
determine a slow-moving metric based on the heartbeat data during a first period of time;
determine a fast-moving metric based on the heartbeat data during a second period of time, the second period of time being less than the first period of time; and
determine a risk level of a health event for the patient based on the slow-moving metric and the fast-moving metric.

16. The method of claim 13 further comprising:
normalizing the plurality of sampled points for each respective heartbeat interval of the plurality of intervals.

17. The method of claim 13 further comprising:
determining a comparison value for each sample point parameter by comparing an absolute value of the difference between the slow-moving average and the fast-moving average for each sample point parameter to a risk threshold; and
determining the risk level of the health event for the patient based on the comparison values for each sample point parameter.

18. The method of claim 17 further comprising:
determining the risk level of the health event for the patient based on a number of consecutive parameter points having the respective comparison value above or equal to the comparison threshold.

19. The method of claim 17 further comprising:
determining the risk level of the health event for the patient based on an amount of the comparison value.

20. The method of claim 13, wherein the plurality of heartbeat intervals are obtained at a respective particular time of day over a plurality of days.
